(19) United States
(12) Patent Application Publication
(10) Pub. No.: US 2009/0143842 A1
(43) Pub. Date: Jun. 4, 2009

(54) Phototherapy Treatment and Device for Infections, Diseases, and Disorders

(76) Inventors: William E. Cumbie, Yorktown, VA (US); Douglas B. Juanarena, Blacksburg, VA (US)

Correspondence Address: MEREK, BLACKMON & VOORHEES, LLC, 673 S. WASHINGTON ST., ALEXANDRIA, VA, 22314, US

(21) Appl. No.: 12/263,501

(22) Filed: Nov. 2, 2008

Related U.S. Application Data
(60) Provisional application No. 60/984,831, filed on Nov. 2, 2007.
(60) Provisional application No. 60/988,837, filed on Nov. 19, 2007.
(60) Provisional application No. 61/051,433, filed on May 8, 2008.
(60) Provisional application No. 61/051,394, filed on May 8, 2008.
(60) Provisional application No. 61/061,678, filed on Jun. 16, 2008.

(51) Int. Cl. A61N 5/06; A61B 5/00

(52) U.S. Cl. 607/88; 600/365

(57) ABSTRACT

A device to treat infections, diseases, and disorders. The device can be used to treat part of a larger organism such as using the device to kill cancerous cells in humans or animals or to kill parasites. The invention can also may be used to treat fungal and bacterial nail infections of the hands and feet which are difficult to treat with oral and topical drugs.

### 1 CROSS REFERENCE TO RELATED APPLICATIONS

This application claims the benefit of U.S. Provisional Application Ser. No. 60/984,831, filed Nov. 2, 2007, Ser. No. 60/988,837, filed Nov. 19, 2007, Ser. No. 61/051,433, filed May 8, 2008, Ser. No. 61/051,394, filed May 8, 2008, and Ser. No. 61/061,678, filed Jun. 16, 2008. Each of the aforementioned applications is incorporated herein by reference.

### 2 BACKGROUND

### Field of Invention

This invention relates to preventing and treating infections, diseases, and disorders using phototherapy

### 3 BACKGROUND OF INVENTION

### Objects and Advantages

This invention according to one aspect reduces or eliminates the need for antibiotics.

This invention can prevent and treat infections, diseases, and disorders.

This invention can prevent nail fungal and bacterial infections

This invention can treat nail fungal and bacterial infections.

This invention can treat infections such as leishmania and MRSA skin infections which are often difficult or impossible to treat with antibiotics.

This invention can be used to treat wounds

The invention can be used to inactivate and/or kill MRSA organisms—it can be used preventatively or to treat an infection caused by these organisms.

This invention can be used for nasal treatment of nares to reduce MRSA.

This invention can be used to treat disorders such as psoriasis and cancer

This invention can be used externally, in body cavities, and internally

This invention can be used on humans and on animals

Each object of the invention need not be met by each and every embodiment of the invention. Where the term “the invention” or “this invention” is used, the term should used to limit any embodiment of the invention as incorporating each and every enumerated feature, aspect or benefit of the invention.

Please note that none of the information contained in this application should be considered medical advice for any person or any circumstance, and a licensed, qualified physician should be consulted prior to attempting any procedure listed herein.

### 4 SUMMARY OF INVENTION

One aspect of the invention disclosed is a device to treat infections, diseases, and disorders. In addition, the invention can be used to treat part of a larger organism such as using the device to kill cancerous cells in humans or animals or to kill parasites. The invention can also specifically be used to treat fungal and bacterial nail infections of the hands and feet which are difficult to treat with oral and topical drugs.

The invention is especially important since drug resistant organisms such as Methicillin-resistant Staphylococcus aureus (MRSA) are a significant and growing problem. Approximately 30% of people harbor MRSA organisms, which contributes as a reservoir of infection, should there be a break in the skin that MRSA can colonize. Also, if MRSA is in the nasal passageways, where it is frequently found, then it can delve deeper into the respiratory tract and cause problems. One way to lower the reservoir of possible infection is to eliminate or reduce MRSA in the nasal passages. There have been attempts which were partially successful at reducing MRSA in the nose by use of an antibiotic ointment. This was sometimes followed up by attempting to recolonize with a more benign strain such as 502A S. aureus. An embodiment of this invention is directed to more effectively reducing or eliminating MRSA in the nostril area to help reduce the chance of a person passing on MRSA by sneezing, etc.

The features and advantages described in specific embodiments outlined in this summary of the invention may be utilized in any of the other embodiments and should be considered as specifically incorporated into all other embodiments unless such incorporation is impracticable or explicitly excluded.

All embodiments of the invention may use germicidal light to prevent and treat infections, diseases, and disorders of the body. Germicidal light is defined in this application as light between 100 nm and 1 mm capable of one or more of the following: inactivate or destroy organisms or cells that are undesirable, condition the treated area to resist or destroy organisms or cells that are undesirable, or that can be combined with one or more other treatments such as medications or other energy treatments to inactivate or destroy organisms or cells that are undesirable.

A variety of germicidal lights may be used to prevent and treat infections, diseases, and disorders, including, but not limited to, UVC, pulsed light that is rich in UVC and contains synergistic wavelengths, UV wavelengths with germicidal characteristics, visible wavelengths with germicidal characteristics, infrared light with germicidal characteristics, and combinations thereof.

Embodiments of this invention may use a variety of germicidal light including but not limited to UVC, pulsed light that is rich in UVC and contains synergistic wavelengths, germicidal light that has been enhanced by using heat or other means to make it more effective, etc. Infrared light may also be used due to its ability to penetrate more deeply and also generate heat which may also help in treatment of diseases, infections, and disorders. A multiplicity of lights can also be used to obtain the most desirable overall spectrum for treatment. In addition, these lights can be staged so they can be sequenced on and off at specified times to provide proper treatment. For example, infrared light could be applied at a high level early in treatment to heat an area and then reduced to maintain a specific temperature during treatment while other light such as UVC is applied once the temperature is reached. This would permit the UVC to damage the organism while the heat generated continues to stress the organism and disrupt cellular processes thereby preventing the organism from repairing genetic damage.

Germicidal light can be monochromatic (such as low pressure mercury or monochromatic plasma or LEDs) or wide spectrum (such as xenon, high pressure mercury, and wide spectrum LEDs). Light can be supplied by a number of sources including low, medium, and high pressure mercury, xenon, deuterium lamp, plasma lamps, surface discharge lamps, LEDs (including organic LEDs), lasers, etc. These light sources could be used in combination with one another or alone. The light can be applied to the inside of a restricted areas such as the nasal passageways by use of small lights that will fit inside the nasal cavity or it may be generated outside the small area and directed into the area to be treated by a light guide or other means. Undesirable light can be minimized or eliminated by the use of filters, reflectors, and other means. For example, if it was desired to minimize or eliminate UVB to prevent sunburning, a notch filter that would eliminate or significantly decrease this bandwidth could be used. Similarly, excessive infrared light could be filtered from a xenon light source by use of mirrors or filters.

Pulsing of light can also be used to condition the light so that it is more germicidal. For example, if a xenon flash lamp is pulsed it will emit considerably more UVC than if it is operated continuously. Additionally, the stronger the pulse (i.e. the higher the pulse density) of the lamp the more UVC will be generated. When a pulse density of 200 A/cm² is used there is some increase in UVC but most of the energy is still in the infrared (IR) range. As the charge density is increased tenfold to 2,000 A/cm² there is significantly more UVC than at 200 A/cm² but there is still more IR. However, increasing the charge density further to 6,000 A/cm² increases the UVC even more and eliminates most of the IR. Pulsed light is also better able to penetrate certain structures such as cells better than the equivalent quantity of non-pulsed light and this capability is enhanced as the pulse is shortened.

Pulse widths are another important parameter in treatment. The shorter pulse width are able to penetrate some structures better—especially cells. These shorter pulse widths can also apply extremely high instantaneous heat and power while still not raising the average power applied to an area. For example, a 1 microsecond pulse every second can have an instantaneous peak energy of 1,000,000 times what the same amount of average energy of a non-pulsed continuous light. Pulse widths of 1 microsecond to 50 milliseconds are particularly contemplated as an aspect of this invention as well as shorter pulses in the 1 to 1,000 femto, 1 to 1,000 pico, and 1 to 1,000 nanosecond range. A preferred variant of this pulse range is that the pulses produce light in the ultraviolet and visible range between 200 nm and 700 nm of which at least 1% is in the range of 200 nm to 280 nm and 10% is in the range of 200 nm to 400 nm.

In addition to the ability of highly pulsed light to penetrate better than non-pulsed light, pulsed light also has a continuous range of light some of which are absorbed by specific cellular structures while others are not. When this light is applied in very short pulses several of these wavelengths may be absorbed by different cellular structures and result in near instantaneous heating of small portions of the cell which then cause the inactivation or destruction of the cell. This is a specifically contemplated advantage of the use of pulsed light compared with non-pulsed light.

Another significant advantage of wide spectrum light (pulse or non-pulsed) is that it can also generate heat and produce erythema on the skin better than UVC light alone. The heat and erythema act to increase blood flow in the area being treated thus permitting the natural defenses of the body to act to help clear the infection itself Increasing the blood flow at the epidermis can significantly help the body successfully attach any infection or foreign cells. Thus while the heat generated during treatment of an area may not be enough to kill the cells outright, the byproduct of this general heating is to stimulate the body's defenses and effectively treat the infected area. This is a specifically contemplated advantage of the use of wide spectrum light compared with light that is monochromatic or that only has a several narrow bands output.

Additionally, other wavelengths that are not as germicidal can be used to synergistically improve the treatment of infections, diseases, and disorders. In addition to the use of synergistic light, how the light is applied such as the use of coherent light (lasers) and pulsing (intensely pulsed light) can also make light that is not normally considered germicidal to act in a germicidal manner. Thus lasers and intensely pulsed light can be used to treat infections and diseases where the same light in a continuous, incoherent form would not be effective in treatment. Germicidal light may be coherent or incoherent, pulsed or continuous, or a combination thereof.

Undesirable light wavelengths can be minimized or eliminated by the use of filters, reflectors, and other means. Other means can also be used to prevent generation of light in the non-desired range or to filter it out at the light or before it reaches the user.

A preferred embodiment of the method to prevent and treat infections, diseases, and disorders using wide spectrum light. A variant of this embodiment would use wide spectrum light that is pulsed, preferentially this light would be highly pulsed so that it has a significant amount of UVC generated. The pulse density of the light could be from 10 A/cm² to more than 10,000 A/cm². In this embodiment, charts of precalculated dosages can be used as guides to the amount of light that is to be delivered to treat a certain disorder, infection, or diseases. While the light is preferentially delivered is wide spectrum highly pulsed light any other light or combination of lights generating suitable spectrums can be used.

Another preferred embodiment of treatment is the use of germicidal light and thermal energy to treat infections, diseases, and disorders. In this embodiment, charts of precalculated dosages can be used as guides to the amount of light that is to be delivered to treat a certain disorder, infection, or diseases. While the light is preferentially delivered is a light emitting primarily UVC (such as a low pressure mercury lamp) in combination with a light emitting primarily IR (such as an LED) any other light or combination of lights generating suitable spectrums can be used.

In another preferred embodiment thermal energy is provided by infrared light. Additional embodiments may use other forms of thermal energy including heat generated by radio waves, ultrasound, microwaves, conductive heat, etc.

Visible light in the range of approximately 480 nm to 560 nm can also provide synergistic wavelengths particularly if two photons (termed two-photon absorption) are absorbed simultaneously to provide light having properties of the more germicidal light in the UVC bandwidth of 240 nm to 280 nm. For example, two photons of 500 nm can be absorbed at the same time and have the effect of a 250 nm photon. It should be noted that it is not required that both photons be of the same wavelength to have two photon absorption—i.e. a 550 nm and a 650 nm photon can be absorbed simultaneously and act as a 250 nm photon. This light band can actually extend from 400 to 600 nm since although light in the bandwidth of 240 to 280 nm has been shown to be most germicidal, light from 200 nm to 300 nm is also germicidal and can be particularly effective on some organisms and in some situations. Two-photon absorption occurs when a molecule absorbs the two photons simultaneously and this raises it energy state in the same manner as absorption of a photon of an equivalent shorter wavelength. Two-photon absorption is several orders of magnitude less frequent than single photon absorption. However, its occurrence increases with the square of the number of photons produced. If the light is highly pulsed such that it has high peak power delivered over a very short time there will be significantly more two-photon absorption than if the same power was delivered at a constant rate. Thus, if the power is delivered in a microsecond pulse (1×10⁻⁶) it will increase the occurrence of two-photon absorption by an order of 1×10¹². If the pulse is shortened even further to the femtosecond level then this process becomes much more significant. Thus a femtosecond laser operating in the range of approximately 400 nm to 600 nm can effectively generate germicidal light to treat infections and disorders. The light may be supplied by one or more monochromatic sources, wide spectrum light, or a combination of the two. Use of highly pulsed light in range of approximately 400 nm to 600 nm to generate germicidal light is one embodiment of the disclosed invention.

In a variant of this preferred embodiment the light may be in the range of 600 nm to 900 nm and triple absorption could provide properties of 200 nm to 300 nm light that can act in a germicidal manner. Though the yield of triple photons would be much lower than two-photon absorption, the light could penetrate much more deeply due to its longer wavelength and thus could be effective to treat diseases, infections, and disorders.

In a preferred embodiment wide spectrum pulsed light with short pulse widths and high instantaneous peak current density creates enhanced penetrability and effectiveness due to the use of two-photon and three-photon and other multi-photon absorption. The longer wavelengths are able to penetrate more deeply and once they do they can combine with other photons to act in an effective germicidal manner.

In a preferred embodiment, germicidal light may be used with other types of treatment such as medications, temperature changes, etc. to increase the efficacy. Use of synergistic treatments may occur prior to, during, or after the application of light or a combination thereof. In one preferred embodiment the temperature of the area treated is raised 10 degrees C. for ten minutes before treatment and again after treatment thus stressing the organisms and interfering with their ability to repair damage caused by light treatment. An alternative embodiment has the area being treated cooled by 10 degrees C. or more for ten minutes prior to treatment and then has the temperature raised during treatment from the optical energy of the treatment. Another preferred embodiment is a cover for the area being treated that can maintain an elevated temperature in the area being treated for a substantial time thereafter. This may be accomplished by external heating by using an insulated covering that retains the heat generated by the body. Cooling of an area could also stress an organism and is also called out as a treatment as well as variations in cooling and heating. In one embodiment a small thermoelectric device that can both heat and cool is attached to the area being treated and a series of alternating temperatures is used to further stress the organisms and to promote stimulation of tissues for healing.

In another preferred embodiment medications can be used in addition to light treatment. For example, after a phototherapy treatment for fungal nail infections, antifungal topical medication can be used on the nails. This could increase the efficacy of the treatment and the phototherapy treatment could desiccate the nails and make it easier for the antifungal compound to permeate the nail. Similarly, antifungal medications could be taken orally in conjunction with phototherapy treatment.

Additionally, another preferred method of treatment involves delivering germicidal light other methods over a long period of time instead of at one or several specific treatment times. For example, a dosage of UVC to treat fungal nails could be delivered over a several day period of time but use of a small LED affixed to the nail that was being treated. This method of treatment may be used as a separate treatment or as an adjunct to other treatment.

Germicidal light may be delivered by a device that can be preprogrammed to deliver a fixed dose of light. It may have several such settings to cover a variety of situations. The device may have preprogrammed settings consistent with the dosage ranges described earlier in this application. It may also be programmable for a custom dosage. The programming can also be used to turn off the light for set periods of time to prevent heat buildup in the unit or in the area being treated or for other reasons. Heat sensors, voltage sensors or other safety equipment can also be added to protect the user and/or equipment.

The device can have a dosage as low as 5 mJ/cm² of UVC to as high as 100 J/cm² of UVC or higher. An example of a preferred dosage would be between 1 J/cm² and 10 J/cm² of UVC to treat mild to moderate fungal nail infections using a series of four treatments spaced on week apart. This could be preferentially delivered using a broad spectrum pulsed light delivering a total dosage of light between approximately 12 J/cm² and 4,800 J/cm². Overall light germicidal light dosages may vary between 5 mJ/cm² to approximately 20,000 J/cm². It should be noted that the number of treatments for mild to moderate onychomycosis can be reduced to one dosage of between 4 J/cm² to 40 J/cm² if that dosage can be delivered with the same effectiveness as four smaller doses.

The device may have a light source that is preferably contained in a light module electrically connected to the treatment device. The light module may contain the light source and other electrical components for converting, rectifying or stepping up or down the voltage to operate the light. The module may also may also contain quick connect connectors necessary to connect the light to the treatment device and may include protection circuitry to simply the safe removal and installation of the light in the device. The module may also protect the user from the heat generated from the light source. Additionally, different modules may be substituted in that contain different permutation of lights, for example one module may contain multiple lights, lights of different wavelengths, filters, or heating elements, etc., necessary for different types of treatments.

In one embodiment the device substantially encloses the area being treated and lowers the level of light escaping the enclosure to acceptable levels.

The device may include sensors or other detectors to prevent operation of the device if it is not properly positioned for treatment. May start on insertion of foot or hands, etc. by pressure trigger. Sensors may be place outside the treatment area to detect light escaping the treatment area. The sensors may cause the device to shut down or cause an alert if electromagnetic waves are escaping the internal cavity of the device. Additionally, the device may cooperate with sensors external to or remote from the device, such as eye protection on a patient. If sensors on the eye protection receive a certain level of radiation, then the device may be shut down. The sensor could be inside or outside of the eye protection. Other sensors are also envisioned. The device may require a code or other authorization to be entered by key pad, key card, biometric sensor input, etc., to prevent the unauthorized operation of the device.

One embodiment of the preferred device uses pulsed light during treatment. The device uses air cooling for the lamp and force air circulation to keep area being treated from becoming uncomfortable. The device has an average electrical use of 100 to 500 watts during treatment. However, the average electrical use can vary from 10 watts to 10,000 watts or more during treatment.

In a preferred embodiment, total treatment time is approximately 10-20 minutes with the lamp flashing at 12 Hz for 5 seconds followed by a 5 second pause. Pulse voltage is approximately 1000 volts. In a variant of this embodiment the pulse voltage could be increased to 10,000 volts thus decreasing the infrared component of the light substantially and permitting treatment to be provided in a 10 minute time frame with a lamp flashing at 12 Hz without pauses. Similar variants could be used by one skilled in the art and programmed into the device. In a preferred embodiment that device would have controls that allowed a multiplicity of simple and complex flashing algorithms to be programmed for treatment of a variety of conditions.

In the preferred embodiment the following light dosage for the treatment of mild to moderate onychomycosis is provided per 10-20 minutes of treatment time.

UVC (100 to 280 nm)—5.3 J/cm²
UVB (280 to 320 nm)—8.3 J/cm²
UVA (320 to 400 nm)—25.8 J/cm²
Visible Light (400 to 750 nm)—177 J/cm²

The method or device can be used to treat other areas of the body besides skin and nails including wounds, body cavities, organs, glands, appendages, etc. For example a device could be used to treat cancer in the lungs, breast, or prostate via the use of a light guide or an optical system that was small enough to insert into the body. Similarly, the device could be used to treat gingivitis of the gums in the oral cavity.

The device to treat infections and disorders can use a special tip that is disposable. Alternatively the tip could have a disposable cover on it. Another alternative is that the tip could be easily sterilized between uses. The tip could also direct or focus the light as desired. Alternately, the tip could be used to evenly diffuse the light. The tip could be coated or fabricated so that it filtered out undesirable wavelengths. The tip could be fitted over fiber optic tip or other light source. The tip could be interlocked with light so that light cannot be triggered until tip is on properly (keeps light from reaching the eyes).

The device could be programmed or preprogrammed to deliver a fixed dose of light. It may have several such settings to cover a variety of situations. It may also be programmable for a custom dosage. The programming could also cover if the light should turn off for set periods of time to prevent heat buildup in the unit or in the area being treated or for other reasons.

The device could be portable. It could be completely contained in a unit sized to be held in a hand or it could have a separate unit that could be set on a table with a cable (light guide or power cable) extending to a hand unit. The device could be rechargeable or run off batteries thus obviating the need for a power cord when in operation. The device could have a charging cradle to charge it between uses. The device could use supercapacitors instead of batteries to provide for rapid recharging and high power in the handheld unit.

In a preferred embodiment it is possible to vary the voltage of the pulses from 100 to 30,000 volts, the current density from 10 amps to 50,000 amps/cm² and the pulse rate from 1 Hz to 1200 Hz or more. In the preferred embodiment it is possible to have the device pulse for a set amount of time and rest for a set amount of time. For example, the device can pulse for 5 seconds on and 5 seconds off or for 60 seconds on and 30 seconds off throughout the treatment time, depending on for example, the needs of the patient or to preventing overheating of the device.

Pulse widths of 1 microsecond to 50 milliseconds are particularly contemplated as part of this invention as well as shorter pulses in the 1 to 1,000 femto, 1 to 1,000 pico, and 1 to 1,000 nanosecond range. A preferred variant of this pulse range is that the pulses produce light in the ultraviolet and visible range between 200 nm and 700 nm of which at least 1% is in the range of 200 nm to 280 nm and 10% is in the range of 200 nm to 400 nm.

In the preferred embodiment the light would be housed in a removable module to facilitate maintenance. In a variant of this embodiment, the module could include other items requiring regular replacement such as the optically transparent window (and any coating of the window), the ozone filter, etc.

In another preferred embodiment it is possible to program a sequence of pulse pauses using for example the key pad or other selector. By way of example, in a 15 minute overall treatment the lamp may pulse on for 60 seconds and off for 60 seconds for the first 6 minutes and then pulse 5 seconds on and 5 seconds off for the rest of the treatment (9 minutes). The variations in pulses and times on and off can vary in a complex pattern if desired. For example, the treatment could start with pulses for one minute at 120 Hz and 1,000 amp current density, then 10 minutes at 10 Hz and 6,000 amp density with 5 seconds of operation and 5 seconds of pauses, and then finish with 10 seconds of 60 Hz pulses at 10,000 amp current density.

In another preferred embodiment the pulsing and pauses between pulses can be controlled by an external input, for example the surface temperature of the area being treated. For example the light could pulse until the nail had a surface temperature of 80 degrees F. and then could alternate pulses and pauses to maintain that temperature throughout treatment. Or the light could pulse until the patient pushed a button which would cause it to pause until the patient pushed the button a second time.

In another preferred embodiment the device is similar to the first embodiment but is composed of two units, one which contains the power module and one which has the light. A variant of this embodiment is that one power unit can be used to power multiple light units.

In another preferred embodiment the device is similar to the first embodiment but is composed of multiple light units which can operate at the same time or independently. The lights may be enclosed in the light module or placed separate. Mirrors or other reflectors or reflective surfaces may be provided to direct light or to enhance the effect of the light. For example, an infrared light could come on to preheat the nail to the desired temperature and then could shut off or continue on when a second light providing UV is used.

In another preferred embodiment the device is similar to the first embodiment but has a means to detect if the light chamber is closed properly so that excessive light does not escape the chamber. One way this could be accomplished is with one or more photodetectors that verify low light levels in the chamber when the light is not on thus indicating light from the outside cannot enter (and light in the chamber cannot escape).

In another preferred embodiment the device is similar to the first embodiment but has means to measure the amount of light actually delivered to the area being treated. The results of the measurement may be used to alter the treatment, such as by changing the overall treatment time to apply the correct dosage, or to turn on, adjust or provide supplemental treatment means in addition to or in place of the main treatment light. For example, when using two lights, if the first light is not providing sufficient radiation a second light may be turned on. Or the number or duration of pulses may be increased in, regardless of the number of lights.

In a preferred embodiment this device could be used to treat skin cancer, warts, etc. with the area surrounding the cancer, warts, etc. masked off and high dosages of light used to inactivate and kill the cancerous cells, viruses causing warts, etc.

In a preferred embodiment the area surrounding the area to be treated may be masked and the mask may remain in place for several treatments. The mask may have a medication that decreases any pain associated with the treatment either during or after the treatment and it may be left in place for a period of time as necessary.

In another preferred embodiment the device is similar to the first embodiment but has interlocks to prevent the unit from operating if the unit is not correctly positioned. This can be done in a number of ways such as the mask having a special material that can be detected by the device such as a fluorescent dye in the mask, an RFID chip, or other ways that one skilled in the art would use.

In another preferred embodiment the device is similar to the first embodiment but uses water or other fluid for cooling for the power and/or lamp as they generate heat during treatment.

In another preferred embodiment the device is similar to the first embodiment but the area irradiated can be cooled by forced air, chilled air, cool water mist, or other means to cool the surface of the skin.

In another preferred embodiment the device is similar to the first embodiment but uses pneumatic skin flattening to enhance the treatment.

In another preferred embodiment the device is similar to the first embodiment but all energy is emitted in one large pulse of light.

In another preferred embodiment the device is similar to the first embodiment but all energy is emitted in several large pulses of light.

In another preferred embodiment the device is similar to the first embodiment but would be capable of delivering 0.01 mj/cm² to 100 mj/cm².

In another preferred embodiment the device is similar to the first embodiment but would be capable of delivering between 100 mj/cm² and 100 J/cm².

In another preferred embodiment the device is similar to the first embodiment but the voltage pulse could be up to 1 gigavolt.

In another preferred embodiment the device is similar to the first embodiment but the current density per pulse could be up to 1 gigaamp.

In another preferred embodiment the device is similar to the first embodiment but the covering used for the extremity is specially designed to interface with the device. This would permit the covering to also block light from exiting the device. For example, the covering for treating fingernails could be similar to a glove with a square flange at the end near the wrist. The device would have a configuration permitting the square flange to be positioned so that it dovetailed with the device thus preventing stray light from exiting. The device could also be configured so that it would not operate unless such a flange was properly in place.

In another preferred embodiment the device is similar to the first embodiment but includes the provision of heat to the area treated from a supplemental source In another preferred embodiment the device is similar to the first embodiment but includes more than on lamp to provide light to the area being treated.

In another preferred embodiment the device is similar to the first embodiment but would have a number of preprogrammed settings to deliver different doses based on different conditions.

In another preferred embodiment the device is similar to the first embodiment but would have the light (and gas mixture) and its envelope materials selected so that ozone would be generated to aid in treatment.

In another preferred embodiment the device is similar to the first embodiment but would have the light (and gas mixture) and its envelope materials selected so that ozone would not be generated as part of treatment.

In another further embodiment the device has a monochromatic and a wide spectrum light.

In another further embodiment the device is used in conjunction with antibiotics In another further embodiment the device has a small number of discreet germicidal bandwidths such as 254 nm, 240 to 280 nm, etc.

In another further embodiment the light is delivered in one to 100,000 pulses, each having more than 10 mj of UVC

In another further embodiment the light is delivered in one to 100,000 pulses, each having more than 1 J of UV

In another further embodiment the light is delivered in one to 100,000 pulses, each having more than 10 J of combined UV, visible, and infrared light

The preferred embodiments are descriptions of possible combinations of features and are illustrative, not exhaustive. One skilled in the art would recognize that the invention could be practiced in various combinations that include or exclude certain items and these combinations are contemplated as part of this invention. Not every feature need be used in every embodiment.

### 5 BRIEF DESCRIPTION OF DRAWINGS

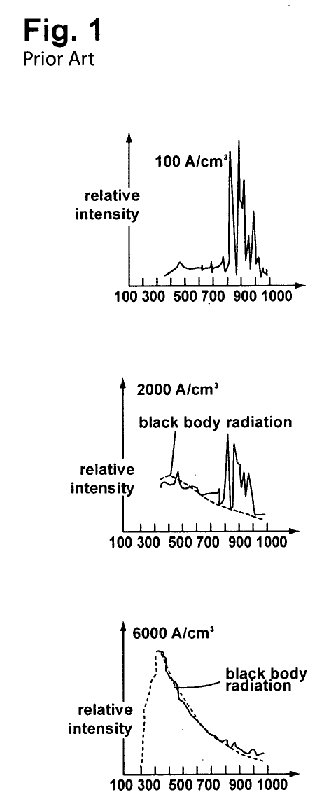

### FIGS. 1-24

FIG. 1—Illustrates three spectrums produced by a xenon lamp as it is pulsed at different current densities. The higher the current density the higher the amount of UVC and the lower the amount of infrared light per pulse.

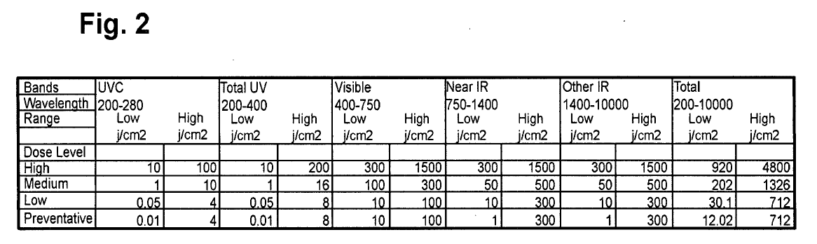

FIG. 2—a chart of light dosage used for prevention and treatment of infections, diseases, and disorders

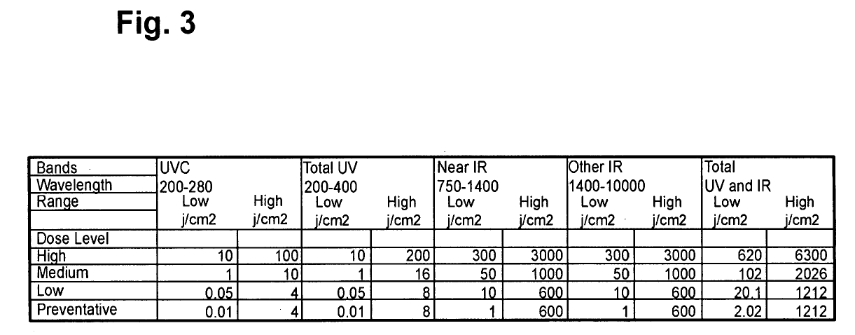

FIG. 3—a chart of UVC and IR light dosages used for prevention and treatment of infections, diseases, and disorders

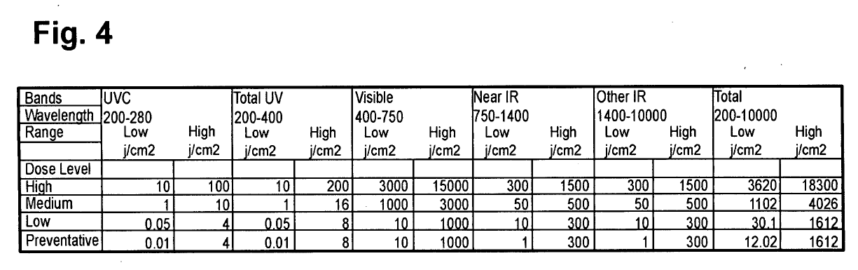

FIG. 4—a chart of laser or highly pulsed light dosages from 400 nm to 600 nm used for prevention and treatment of infections, diseases, and disorders

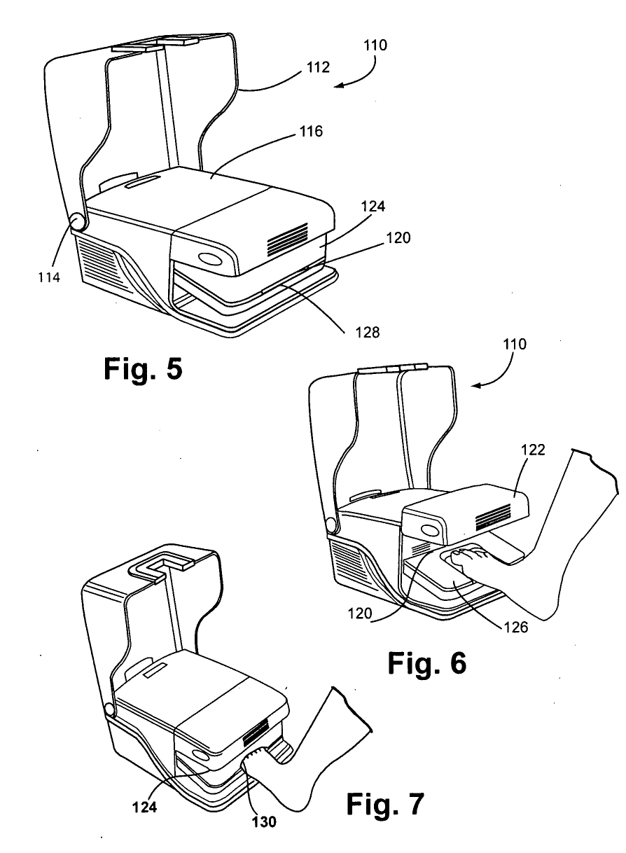

FIG. 5—Device to prevent and treat infections and disorders of the hands and feet

FIG. 6—Device to treat hands and feet in open position

FIG. 7—Device to treat hands and feet partially closed position

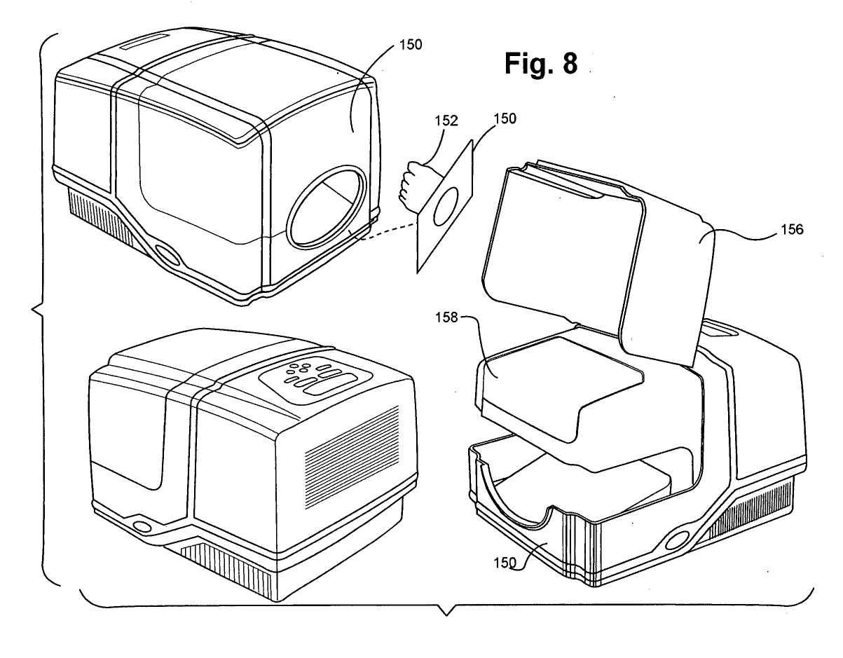

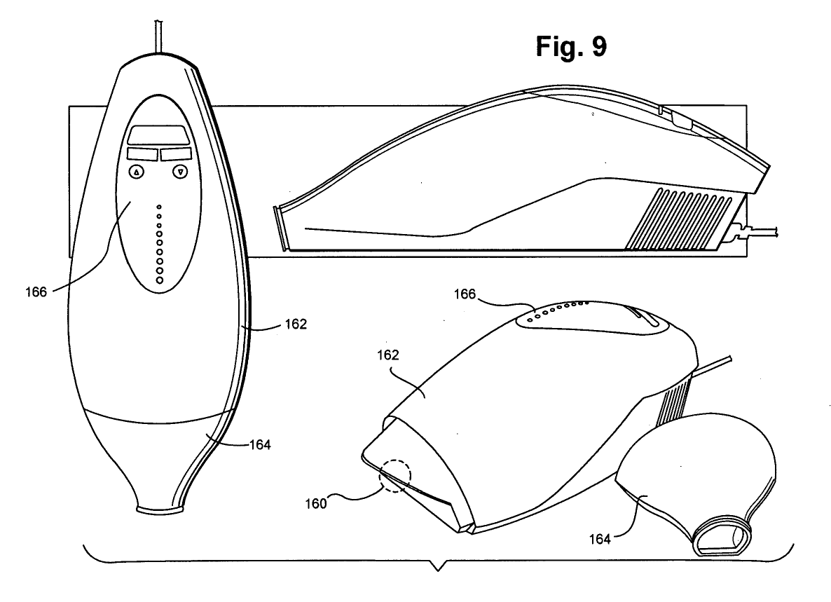

FIG. 8—Device using a opening that can accept a flanged fitting

FIG. 9—Device used to treat one nail at a time

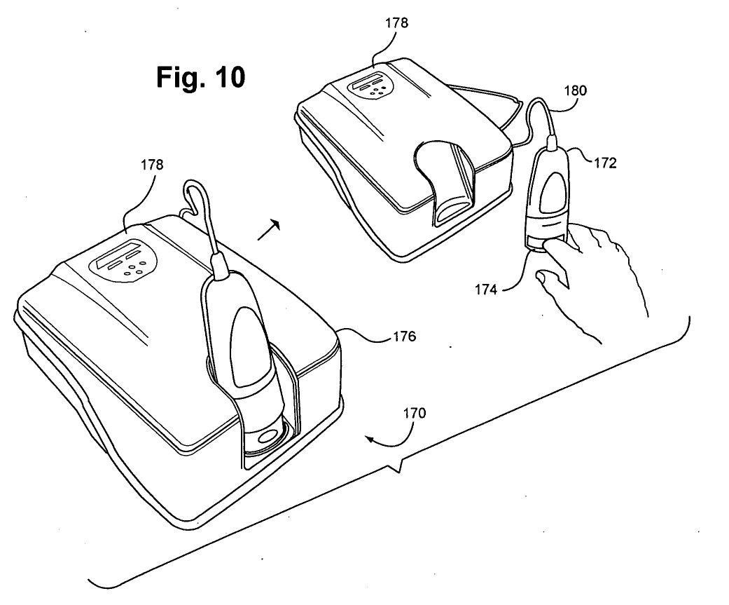

FIG. 10—Device used to treat one nail at a time and can treat skin

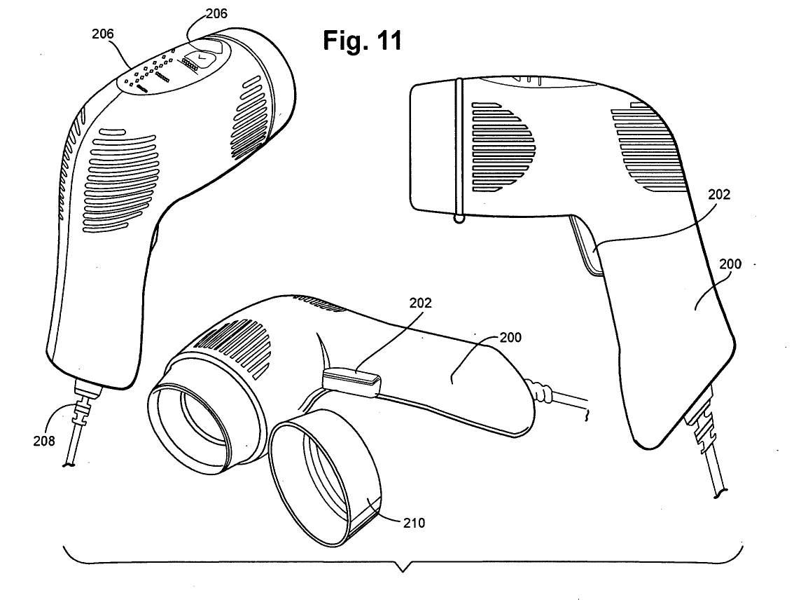

FIG. 11—Device used to treat skin with pistol grip

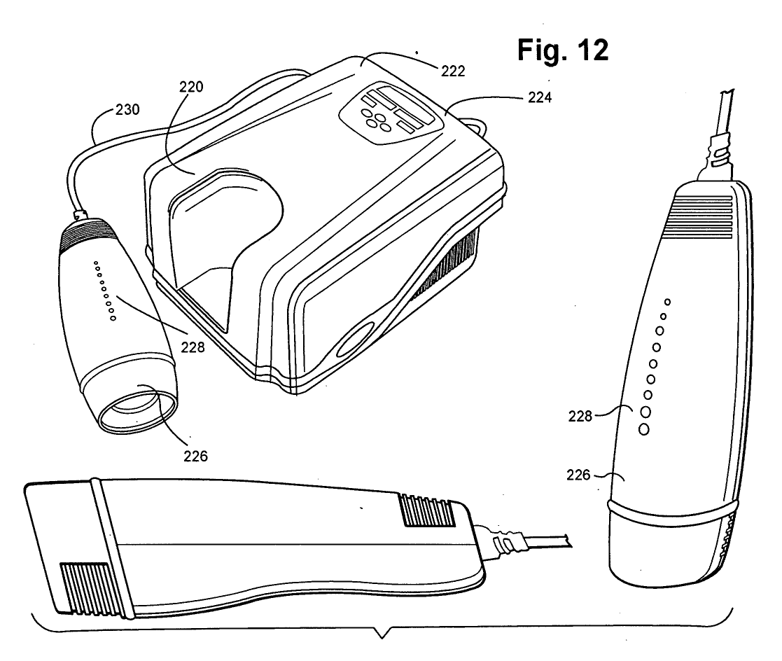

FIG. 12—Device used to treat skin with wand

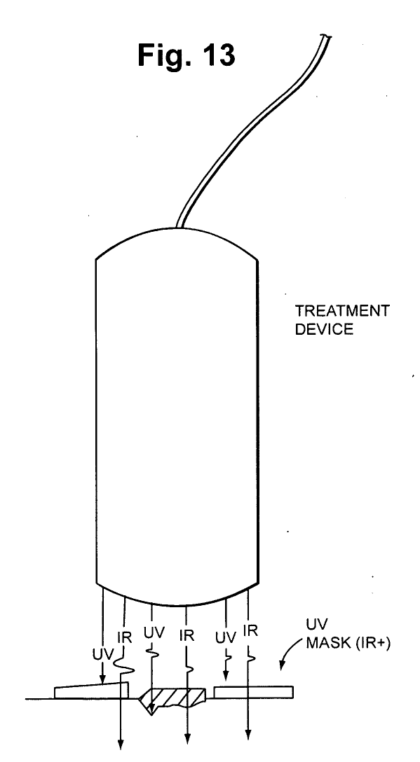

FIG. 13—Covering that screens UV but allows IR to penetrate

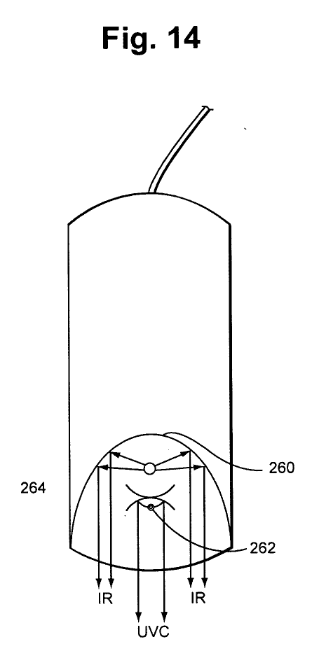

FIG. 14—Light device emitting UVC and IR

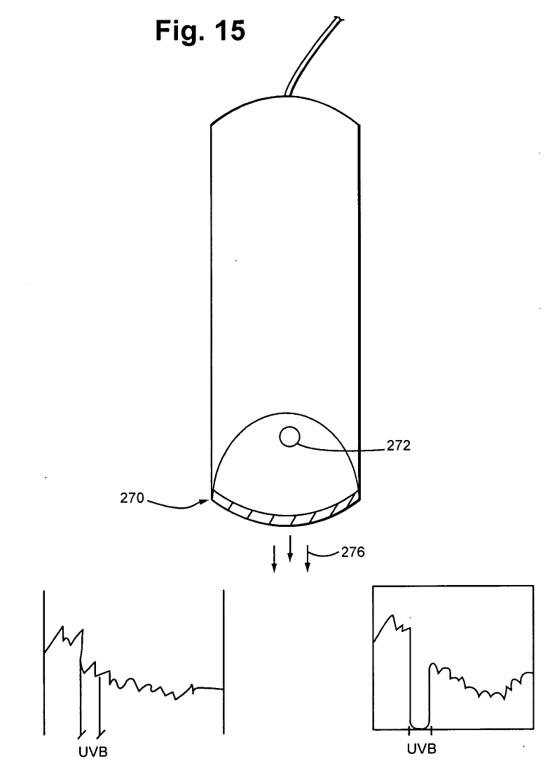

FIG. 15—use of a UVB notch filter

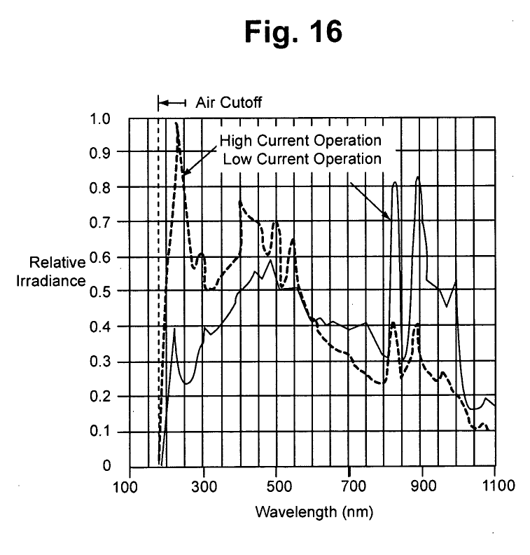

FIG. 16—comparison of high and low current pulsing of xenon lamp

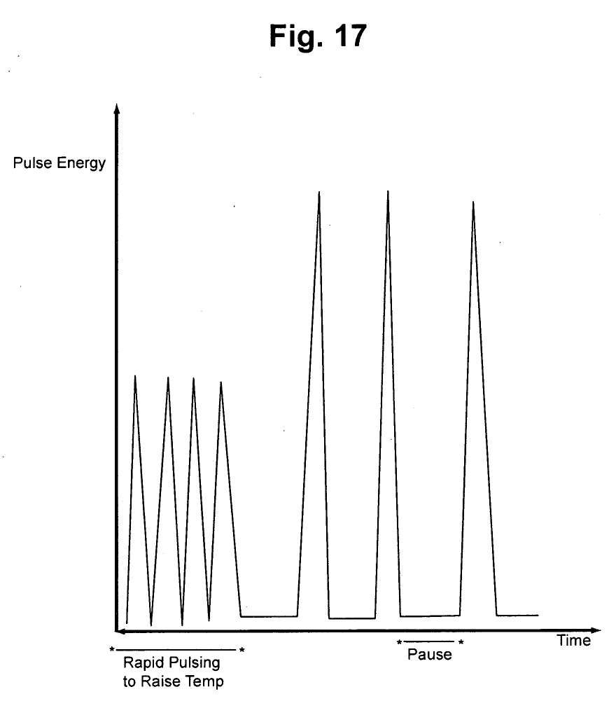

FIG. 17—schematic of pulsing and pauses during treatment

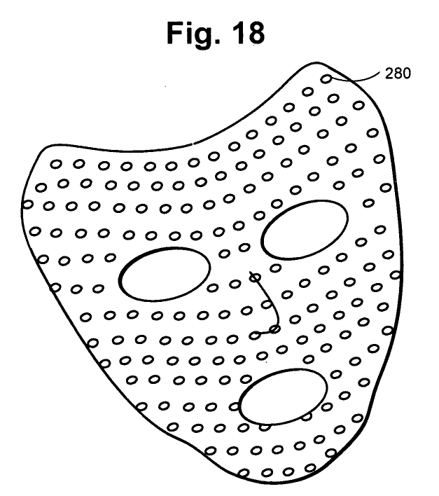

FIG. 18—face mask with small lights

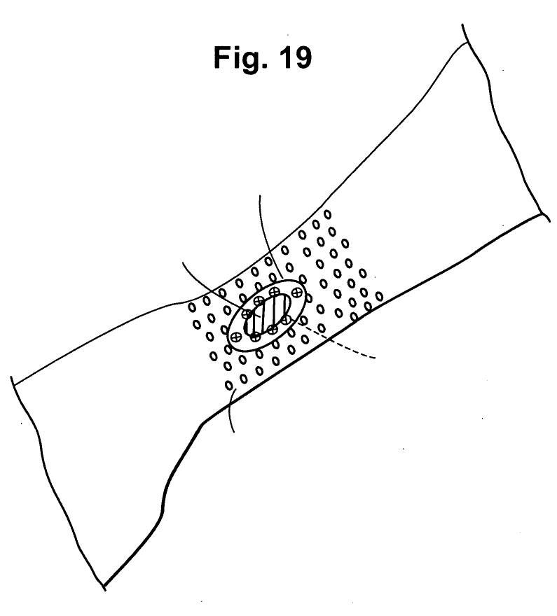

FIG. 19—flexible array with small lights

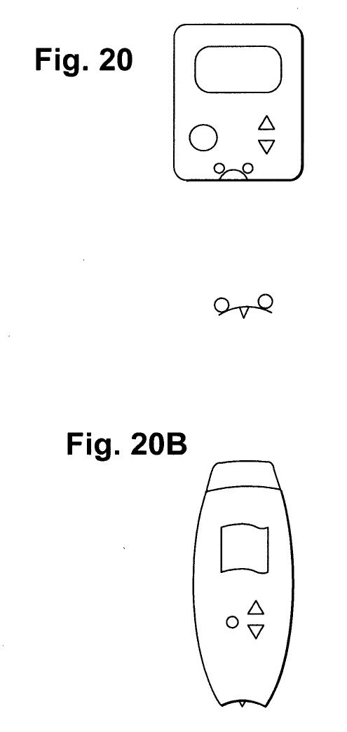

FIG. 20—light integrated into a diabetes blood glucose monitor.

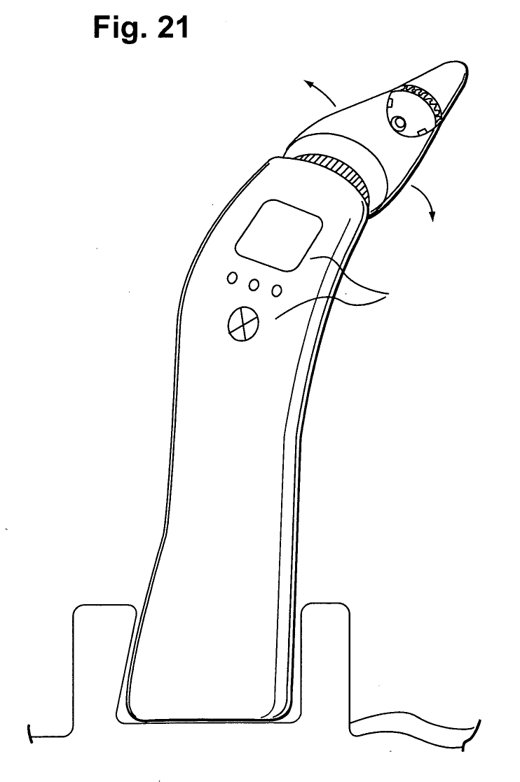

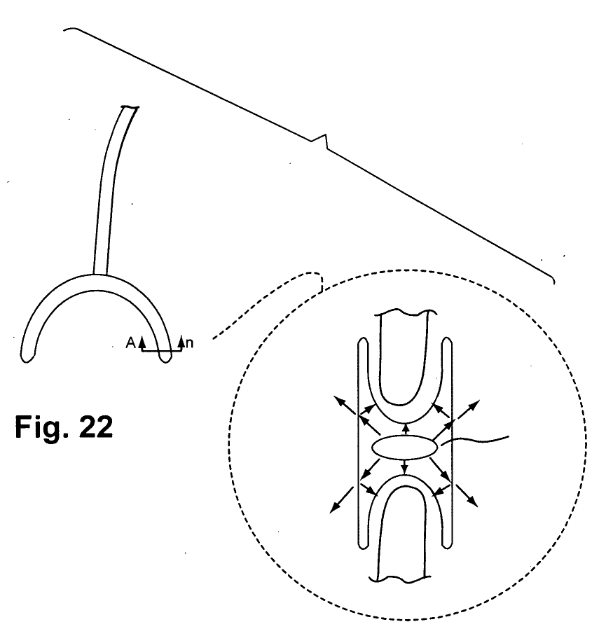

FIG. 21—nostril treatment device.

FIG. 22—mouth treatment device

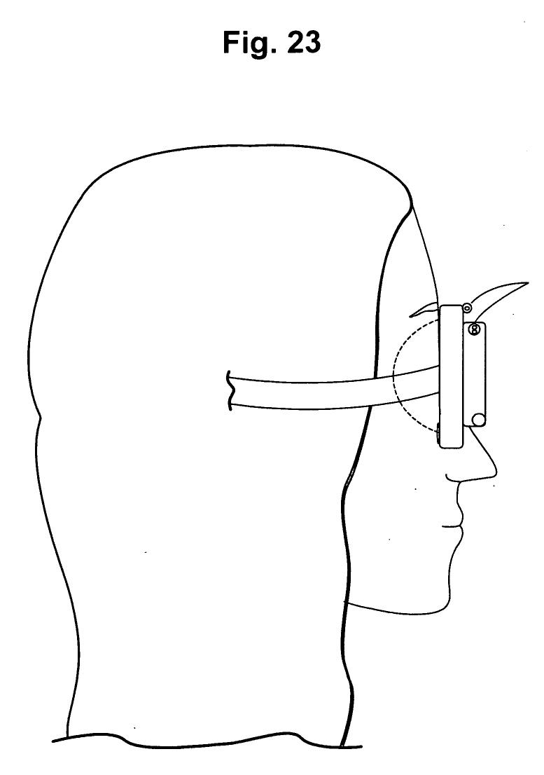

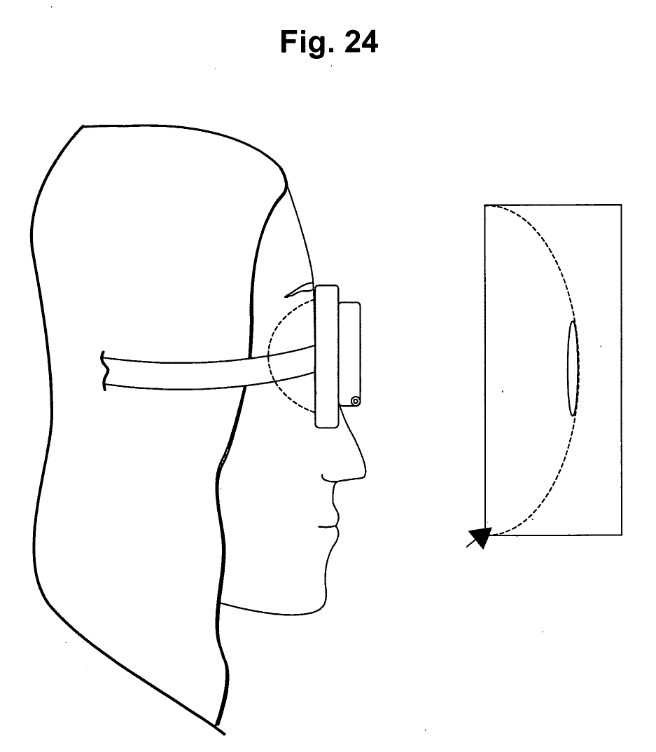

FIG. 23—smart treatment goggles

FIG. 24—treatment goggles interlocked with treatment device

### 6 DESCRIPTION OF PREFERRED EMBODIMENTS

The preferred embodiments are descriptions of possible combinations of features and are illustrative, not exhaustive. One skilled in the art could develop similar combinations that include or exclude certain items and these combinations are contemplated as part of this invention.

In a preferred embodiment, a light treatment is applied to a user, for example to cure toe nail fungus. A light device may be used to provide both the light and to position an area of the user to be treated. As show in FIG. 6, a light device 110 contains a source of light 120. A user can position a hand or foot 121, for example, on a tray 126 to receive a light treatment. A cover 112 may be used to retain light from the source within the device and to keep external light from interfering with the treatment.

In a preferred embodiment pulsing of light can also be used to condition the light so that it is more germicidal. For example, if a xenon flash lamp is pulsed it will emit considerably more UVC than if it is operated continuously. Additionally, the stronger the pulse (i.e. the higher the pulse density) of the lamp the more UVC will be generated. The stronger the pulse, the less infrared that also will be generated. FIG. 1 shows how the spectrum varies with the strength of the pulse. When a pulse density of 200 A/cm² is used there is some increase in UVC but most of the energy is still in the IR range. As the charge density is increased tenfold to 2,000 A/cm² there is significantly more UVC than at 200 A/cm² but there is still more IR. However, increasing the charge density further to 6,000 A/cm² increases the UVC even more and eliminates most of the IR.

The first two of these charge densities has been tested and found to be effective in treating nail & skin infections. The last charge density will also be effective in treating infections and disorders since it not only maximizes UVC but it also greatly increases the number of two-photon absorptions between 400 nm and 600 nm that will mimic a UVC photon and thus further increase efficacy. All three charge densities and those that are between are specifically contemplated as part of this invention. Additionally, densities in excess of 6,000 A/cm² are also specifically contemplated and may be especially useful as one-pulse treatments (or a small number of pulses treatment).

In a preferred embodiment the following modes of operation described may be used by the devices described in this application or they may be used by other devices. Similarly, while the devices described may use the modes of operation described, they may also used modes of operation not described.

### Mode of Operation Using Highly Pulsed Light and UV Light

A preferred embodiment to prevent and treat infections, diseases, and disorders uses wide spectrum light. A variant of this embodiment would use wide spectrum light that is pulsed, preferentially this light would be highly pulsed so that it has a significant amount of UVC generated. The pulse density of the light could be from 10 A/cm² to more than 6,000 A/cm². In this embodiment, charts of precalculated dosages can be used as guides to the amount of light that is to be delivered to treat a certain disorder, infection, or diseases. An example treatment chart is shown in FIG. 2. This dosage chart is used by determining the infection, diseases, or disorder and its level of severity. A precise diagnosis of the infection, disease, or disorder is not required but it can be helpful. The chart provides and overall dosage range for a infection, disease or disorder based on its severity. The categories of light applied can be used together or they can be used separately. For example, if a medium dosage is desired it can be either a dosage from 1 to 10 J/cm² of UVC, or 1 to 16 J/cm² of UV, or 100 to 300 J/cm² of Visible light, or 50 to 500 J/cm² of near IR, or 50 to 500 J/cm² of far IR, or 202 to 1,326 J/cm² of light overall. Thus, treatment can consist of only one of these ranges or a combination of some or all of these ranges. The dosages listed are what is provided to the top of an area being treated (“surface”) during the total course of one treatment. This dosage can be varied between 50% to 400% for treatment of an area and can be provided once or multiple times as necessary. The above precalculated doses can be used or one who is skilled in the art may want to vary the doses according to their own calculations and professional judgment. High dosages may also be used in internal areas of the body which may not have as many nerves and so do not have as strong a pain response. Higher dosages may be preferential internally to treat cancer and other serious infections and disorders. Some of the light in these ranges can also be filtered or screened out. For example, some or all of the UVB can be screened out to prevent or lessen erythema. Similarly, excess IR may be screened or filtered out to prevent excess heat. While the light is preferentially delivered is wide spectrum highly pulsed light any other light or combination of lights generating suitable spectrums can be used.

### Mode of Operation Using UV Light and Thermal Energy

A preferred embodiment to prevent and treat infections, diseases, and disorders uses wide spectrum light germicidal light and thermal energy. In this embodiment, charts of precalculated dosages can be used as guides to the amount of light that is to be delivered to treat a certain disorder, infection, or diseases. An example treatment chart is shown in FIG. 3. This dosage chart is used by determining the infection, diseases, or disorder and its level of severity. A precise diagnosis of the infection, disease, or disorder is not required but it can be helpful. The chart provides and overall dosage range for an infection, disease or disorder based on its severity. The categories of light applied can be used together or they can be used separately. For example, if a medium dosage is desired it can be either a dosage from 1 to 10 J/cm² of UVC, or 1 to 16 J/cm² of UV, or 50 to 1,000 J/cm² of near IR, or 50 to 1,000 J/cm² of far IR, or 102 to 2,026 J/cm² of light overall. Thus, treatment can consist of only one of these ranges or a combination of some or all of these ranges. The dosages listed are what is provided to the top of an area being treated (“surface”) during the total course of one treatment. This dosage can be varied between 50% to 400% for treatment of an area and can be provided once or multiple times as necessary. The above precalculated doses can be used or one who is skilled in the art may want to vary the doses according to their own calculations and professional judgment. Some of the light in these ranges can also be filtered or screened out. For example, some or all of the UVB can be screened out to prevent or lessen erythema. Similarly, excess IR may be screened or filtered out to prevent excess heat. While the light is preferentially delivered is a light emitting primarily UVC (such as a low pressure mercury lamp) in combination with a light emitting primarily IR (such as an LED) any other light or combination of lights generating suitable spectrums can be used.

In the preferred embodiment thermal energy is provided by infrared light. Additional embodiments may use other forms of thermal energy including heat generated by radio waves, ultrasound, microwaves, conductive heat, etc.

### Mode of Operation Using Highly Pulsed Light and Laser Light Between Approximately 400 nm and 600 nm and UV

Another preferred embodiment to prevent and treat infections, diseases, and disorders uses very short pulsed light in the range between 400 and 600 nm with UV light in the range of 100 nm to 400 nm. Alternately the very short pulsed light can be used alone. The shorter the pulse of light the greater the two-photon absorption which generates the equivalent germicidal wavelength (i.e. as two 500 nm photons are absorbed they generate damage to the DNA that is similar to that caused by 250 nm light). In the preferred embodiment the light can be generated by a femto-, pico-, or nanosecond laser. An additional variant can include the additional of thermal energy such as the use of IR, germicidal light and thermal energy to treat infections, diseases, and disorders. In this embodiment, charts of precalculated dosages can be used as guides to the amount of light that is to be delivered to treat a certain disorder, infection, or diseases. An example treatment chart is shown in FIG. 4. This dosage chart is used by determining the infection, diseases, or disorder and its level of severity. A precise diagnosis of the infection, disease, or disorder is not required but it can be helpful. The chart provides and overall dosage range for a infection, disease or disorder based on its severity.

For example, the primary treatment could use highly pulsed visible light in the range of 400 nm to 600 nm with either a laser or non-coherent source or a combination of the two with a total dosage of 1000 to 3000 J/cm² for a precalculated medium dosage. Additionally, adjunct light could also be used with a range of dosages for a precalculated medium dosage of 1 to 10 J/cm² of UVC, or 1 to 16 J/cm² of UV, or 50 to 500 J/cm² of near IR, or 50 to 500 J/cm² of far IR, or 1,102 to 4,026 J/cm² of light overall. Thus, treatment can consist of only one of these ranges or a combination of some or all of these ranges. The dosages listed are what is being provided to the top of an area being treated (“surface”) during the total course of one treatment. This dosage can be varied between 50% to 400% for treatment of an area and can be provided once or multiple times as necessary. The above precalculated doses can be used or one who is skilled in the art may want to vary the doses according to their own calculations and professional judgment. Some of the light in these ranges can also be filtered or screened out. For example, some or all of the UVB can be screened out to prevent or lessen erythema. Similarly, excess IR may be screened or filtered out to prevent excess heat. While the light is preferentially delivered is a light emitting primarily UVC (such as a low pressure mercury lamp) in combination with a light emitting primarily IR (such as an LED) any other light or combination of lights generating suitable spectrums can be used.

In a variant of this preferred embodiment the light may be in the range of 600 nm to 900 nm and triple absorption could provide an equivalent of 200 nm to 300 nm light that can act in a germicidal manner. Though the yield of triple photons would be much lower than two-photon absorption, the light could penetrate much more deeply due to its longer wavelength and thus could be effective to treat diseases, infections, and disorders.

### Mode of Operation Using Synergistic Treatments Including Medications, Temp Changes, Etc.

A preferred embodiment to prevent and treat infections, diseases, and disorders uses germicidal with other types of treatment such as medications, temperature changes, etc. to increase the efficacy. Use of synergistic treatments may occur prior to, during, or after the application of light or a combination thereof. In one preferred embodiment the temperature of the area treated is raised 10 degrees C. for ten minutes before treatment and again after treatment thus stressing the organisms and interfering with their ability to repair damage caused by light treatment. An alternative embodiment has the area being treated cooled by 10 degrees C. or more for ten minutes prior to treatment and then has the temperature raised during treatment from the optical energy of the treatment. Another preferred embodiment is a cover for the area being treated that can maintain an elevated temperature in the area being treated for a substantial time thereafter. This may be accomplished by external heating by using an insulated covering that retains the heat generated by the body. Cooling of an area could also stress an organism and is also called out as a treatment as well as variations in cooling and heating. In one embodiment a small thermoelectric device that can both heat and cool is attached to the area being treated and a series of alternating temperatures is used to further stress the organisms and to promote stimulation of tissues for healing.

In another preferred embodiment medications can be used in addition to light treatment. For example, after a phototherapy treatment for fungal nail infections, antifungal topical medication can be used on the nails. This could increase the efficacy of the treatment and the phototherapy treatment could desiccate the nails and make it easier for the antifungal compound to permeate the nail. Similarly, antifungal medications could be taken orally in conjunction with phototherapy treatment.

### Mode of Operation Using of Combinations Discussed Previously and Low Level Usage

A preferred embodiment to prevent and treat infections, diseases, and disorders includes combinations of any or all of the four modes of operation discussed in this application. Additionally, another preferred method of treatment involves delivering the dosages outlined in the other methods over a long period of time instead of at one or several specific treatment times. For example, a dosage of UVC to treat fungal nails could be delivered over a several day period of time but use of a small LED affixed to the nail that was being treated.

### Preferred Embodiment for the Treatment of Nail Infections of the Hands and Feet

A preferred embodiment of the device can be used to treat nail infections of the hands and feet and comprises a portable housing having a germicidal light and an enclosure that can surround the area being treated. In the preferred embodiment most of the light generated does not escape the enclosure surrounding the treated area. In the preferred embodiment the device can accommodate one foot or hand. In a variant embodiment the device can treat both hands or both feet at the same time.

A variety of germicidal lights can be used, including but not limited to, UVC, pulsed light that is rich in UVC and contains synergistic wavelengths, germicidal light that has been enhanced by using heat or other supplemental or additive treatments to make the overall treatment more effective, germicidal light without UV, etc. may be used. Germicidal light can be monochromatic (such as low pressure mercury) or wide spectrum (such as xenon). Light can be supplied by a number of sources including low, medium, and high pressure mercury, xenon, deuterium lamp, LEDs (including organic LEDs), lasers, surface discharge lamps, plasma lamps, etc. These light sources could be used in combination with one another or alone.

Undesirable light wavelengths can be minimized or eliminated by the use of filters, reflectors, and other means. For example, if it were desired to minimize or eliminate UVB to prevent sunburning, a notch filter that would eliminate or significantly decrease this bandwidth could be used. Other means can also be used to prevent generation of light in the non-desired range or to filter it out at the light or before it reaches the user.

In the preferred embodiment the device can be used to treat one or more nails at a time. Skin and nails that are not being treated can be protected by a covering that is opaque to UV light.

The device can be preprogrammed to deliver a fixed dose of light. It may have several such settings to cover a variety of situations. The device may have preprogrammed settings consistent with the dosage ranges described earlier in this application. It may also be programmable for a custom dosage. The programming can also be used to turn off the light for set periods of time to prevent heat buildup in the unit or in the area being treated or for other reasons. Heat sensors, voltage sensors or other safety equipment can also be added to protect the user and/or equipment.

The device can have a dosage as low as 5 mJ/cm² to as high as 100/cm² of UVC or higher. Preferred dosage is between 1 J/cm² and 10 J/cm² of UVC to treat nail infections. Preferentially delivered using a broad spectrum pulsed light delivering a total dosage of light between approximately 12 J/cm² and 4,800 J/cm².

The preferred embodiments are descriptions of possible combinations of features and are illustrative, not exhaustive. One skilled in the art would recognize that the invention could be practiced in various combinations that include or exclude certain items and these combinations are contemplated as part of this invention. Not every feature need be used in every embodiment.

Referring to FIG. 5, the first preferred embodiment according to one aspect of the invention comprises is a unit with approximate dimensions of 12-in wide by 12-in high by 17-in long weighing approximately 25 pounds. The device is capable of irradiating an area of approximately 2-inches by 4-inches. However, treatment area can vary from approximately 1 square inch to more than 50 square inches. One skilled in the art would appreciate that other sized and configuration units for irradiating larger or smaller areas.

As shown in FIG. 5, the light treatment device 110 according to a preferred embodiment of the invention comprises an outer protective cover for preventing accidental initiation of the treatment and protects electronic components within the housing. In operation, the outer protective cover 112 is opened by lifting the hinged cover about hinge 114 to expose the inner device. The inner housing includes a key pad 116 for programming treatment. Lifting the outer cover also exposes the treatment area 120 and provides access to the light shields.

As shown in FIGS. 6-7, once the cover is opened, the treatment area is accessed by lifting the upper light housing 122 and upper shield 124 to expose a foot tray 126. When the user's foot is in place on the foot tray, the upper light housing 122 is lowered and the upper shield 124 is lowered to prevent light from escaping above the foot. The upper shield 124 may be a foam material or other flaccid or stiff material that conforms to the foot to limit the amount of light escaping around the foot. The material may be preformed to the shape of the foot or may automatically conform to the foot as it contacts the foot. Foam material are preferably as providing comfort to the user while closely conforming to the foot to prevent undue escape of light.

The lower tray may include a foot receiving area 126 may include a cut out or recessed area 128 for receiving the foot. This provides the dual purposes of fixing the foot in the intended area and provides side walls to prevent light from escaping around the side of the foot. Since the light has wavelengths that need to be controlled, it is preferable to limit the amount of light escaping from the intended area. Uncontrolled light could cause sun burn like burns or other desirable effects.

The foot tray could be made of a plastic material and may be stiff or may be provided with padding or other material to make the device as comfortable as possible. The inner space 130 bounded by the upper light housing, upper shield and lower foot tray forms a treatment area 130. A light module is provided at the upper side of the treatment area, though one skilled in the art would recognize that the light could be provided at other areas of the treatment area in place of or to supplement the upper light.

Inserts can be used that mate with or utilize the foot space. For example, an attachment which receives one or more fingers or a hand may be provided that fits within the recess 128 on the foot tray 126. The insert may have appropriate openings and shielded areas or transparent or translucent areas to direct treatment to the hand or fingers as desired. It may also be possible to place a hand or finger on the foot tray using a covering or other shield on the hand or finger to direct light to the appropriate area and keep the light off the undesired area. Other sized treatment areas can be provided or shields can be provided to the treatment area in the open configuration (with the upper shield raised) if appropriate to allow larger tissues, organs or areas to be treated. Shields, curtains or the like may be provided to prevent light from escaping in the open configuration.

The foot tray is preferably provided at or near the base of the treatment device. An extendable foot stay (not shown) may be provided to support parts of the foot, hand, arm or body extending from the treatment area beyond the boundaries of the treatment device. In the preferred embodiment, the foot tray is slightly raised above the base of the treatment device housing, but close enough that the user can comfortably place part of the foot in the machine and part of the foot on the floor or other surface containing the treatment device. Since the treatments may last an extended period of time, it is important for the user to be comfortable during the treatment.

The light source is preferably contained in a light module electrically connected to the treatment device. The light module may contain the light source and other electrical components for converting, rectifying or stepping up or down the voltage to operate the light. The module may also may also contain quick connect connectors necessary to connect the light to the treatment device and may include protection circuitry to simply the safe removal and installation of the light in the device. The module may also protect the user from the heat generated from the light source. Additionally, different modules may be substituted that contain different permutation of lights, for example one module may contain multiple lights, lights of different wavelengths, filters, or heating elements, etc., necessary for different types of treatments.

The foot tray or upper shield may include sensors or other means to prevent operation of the device if a foot or other subject is not in place, or to halt operation if the foot or other treatment subject is removed or if the shield is lifted or not secured. Sensors may be place outside the treatment area 120 to detect light escaping the treatment area. The sensors may cause the device to shut down or cause an alert if electromagnetic waves are escaping the internal cavity of the device. Additionally, the device may cooperate with sensors external to or remote from the device, such as eye protection on a patient. If sensors on the eye protection receive a certain level of radiation, then the device may be shut down. The sensor could be inside or outside of the eye protection. Other sensors are also envisioned. The device may require a code or other authorization to be entered by key pad, key card, biometric sensor input, etc., to prevent the unauthorized operation of the device.

FIG. 8 illustrates a device that has an opening 150 that can accommodate a protective nitrile glove for the foot or hand 152 that has a flange 154 that can fit into the opening 150 thus sealing the opening and preventing light from escaping. This configuration also has the advantage of more precisely positioning the hand or foot being treated. The tips of the nitrile glove that cover the nails to be treated can be removed by cutting while leaving the other tips intact to protect untreated nails.

FIG. 8 also illustrates a smaller cover 156 for the device which lifts to permit the flanged 154 glove 152 to be inserted and then closed for treatment. A smaller removable light unit 158 is also shown. This removable light unit 158 is designed to be replaced without special tools in a manner similar to replacing a toner cartridge for a laser printer.

FIG. 9 illustrates one variation of a preferred embodiment of the device designed to treat one nail at a time. The light 160 is located in the body of the device and the light can be directly focused on the nail directed to the nail by mirrors or reflectors. A nose piece 164 can accommodate one finger or toe and can be removed for cleaning. Simple controls 166 for the device are shown and can be used to set time of exposure, exposure strength, etc.

Another configuration of the device to treat single nails is shown in FIG. 10. A unit 170 may include an internal chamber for treating a patient, but preferably includes a remote unit 172 having an opening 174 for receiving a finger or other digit therein. The remote unit in 172 is shown as having an upper and lower portion slidably adjustable relative to each other to limit or expand the dimensions of opening 174. In this way, the finger or other digit can be comfortably received while disallowing light from a phototherapy treatment to escape around the finger. The device could have an adaptor which has the opening 174 for the digit which can be removed thus permitting the unit to be used to treat skin or other areas. Also, one device may be able to service multiple remote units 172 so that more than one nail may be used at a time. The units could act simultaneously or they may act sequentially thus reducing the average power required by the separate main unit 176. For example, if each individual remote unit 174 requires 12 pulses per second and ten units were plugged in the main unit could pulse 120 times per second with the light being sequentially distributed to each of the ten remote unit 174.

Light produced in the main unit 176 may be channeled through fiber optic or other light guide to the remote unit and applied to the portion of the finger or digit within the remote unit 172.

The device 170 could be preprogrammed to deliver a predetermined dose of light. The unit may have a control panel 178 for selecting the dosage to be applied. Several settings may be preprogrammed to cover a variety of situations or the unit may be adjustable for programming a custom dosage. The programming could also control whether the light will turn off (“rest”) for set periods of time to control the heat generated in order to enhance treatment. The device could also be portable. It could be completely contained in a unit sized to be held in a hand 172 or it could have a separate unit 176 that could be set on a table with a cable (light guide or power cable) 180 extending to a hand unit 172. The device could be rechargeable or run off batteries (not shown) thus obviating the need for a power cord when in operation. The device could have a charging cradle to charge it between uses. The device could use supercapacitors instead of batteries to provide for rapid recharging and high power in the handheld unit.

In a preferred embodiment, total treatment time is approximately 10-20 minutes with the lamp flashing at 12 Hz for 5 seconds followed by a 5 second pause. Pulse voltage is approximately 1,000 volts. In a variant of this embodiment the pulse voltage could be increased to 10,000 volts thus decreasing the infrared component of the light substantially and permitting treatment to be provided in a 10 minute time frame with a lamp flashing at 12 Hz without pauses. Similar variants could be used by one skilled in the art and programmed into the device. In a preferred embodiment that device would have controls that allowed a multiplicity of simple and complex flashing algorithms to be programmed for treatment of a variety of conditions.

Automatic shut off switches may be provided to prevent overheating.

In the preferred embodiment the following light dosage for the treatment of mild to moderate onychomycosis is provided per 10-20 minutes of treatment time with actual light delivery for 10 minutes:

UVC (100 to 280 nm)—5.3 J/cm²
UVB (280 to 320 nm)—8.3 J/cm²
UVA (320 to 400 nm)—25.8 J/cm²
Visible Light (400 to 750 nm)—177 J/cm²
Infrared Light (750 to 2300 nm)—50 to 150 J/cm²
This dosage is what is provided to the top of an infected nail (“surface”) being treated during the total course of one treatment. This dosage can be varied between 50% to 400% for treatment of the average infected nail and can be provided once or multiple times as necessary. This equates to a dosage of approximately 2.5 to 10 J/cm² of UVC.

In a variant of the preferred embodiment the heat generated by infrared light is partially or totally screened out or the current density per pulse is adjusted to be higher to significantly decrease the amount of infrared light generated per pulse.

Much lower dosages in the range of 1% to 50% of this dose can be applied to early nail infections or as a preventative treatment. This equates to a dosage of 0.05 to 2.5 J/cm² of UVC.

In the preferred embodiment it is possible to vary the voltage of the pulses from 100 to 30,000 volts, the current density from 10 amps to 50,000 amps/cm² and the pulse rate from 1 Hz to 1200 Hz or more. Pulse widths of 1 microsecond to 50 milliseconds are particularly contemplated as part of this invention as well as shorter pulses in the 1 to 1,000 femto, 1 to 1,000 pico, and 1 to 1,000 nanosecond range. A preferred variant of this pulse range is that the pulses produce light in the ultraviolet and visible range between 200 nm and 700 nm of which at least 1% is in the range of 200 nm to 280 nm and 10% is in the range of 200 nm to 400 nm. In the preferred embodiment it is possible to have the device pulse for a set amount of time and rest for a set amount of time. For example, the device can pulse for 5 seconds on and 5 seconds off or for 60 seconds on and 30 seconds off throughout the treatment time.

In the preferred embodiment the device contains an ozone filter. The device may utilize forced cooling air and force exhaust to keep the lamp cool and create a comfortable environment of the area being treated. This may especially be true when pre-cooling or post cooling the area heated by the application of electromagnetic energy. It also has a special plate ergonomically designed to accept a foot, a hand placed palm down to treat all nails except, according one preferred embodiment, the nail of the thumb or alternatively, just a thumbnail when the rest of the hand is located under the plate. Alternatively, a second light underneath the tray could illuminate the thumbnail while the main light illuminates the nails of the digits. The plate may be replaceable for different applications or to conform to different body parts. The plate may include shields for various parts such as individual fingers not being treated or may incorporate attachment points for attachable shields. The device could have an optically transparent window between the light and the area being treated which would increase safety and control heat flow. The window could also have a coating to filter or reflect or filter wavelengths of light that are undesirable such as UVB.

In the preferred embodiment the light would be housed in a removable module to facilitate maintenance. In a variant of this embodiment, the module could include other regularly replaced item such as the optically transparent window (and any coating of the window), the ozone filter, etc.

In the preferred embodiment the unit has a plate that helps properly position the area being treated whether it is a foot, a thumb, or a hand. This plate has boundaries for containing or retaining the area being treated such as, for example, a depression. In the preferred embodiment, the unit can also close in a manner that it restrains the extremity being treated and keeps it from moving accidentally.

In another preferred embodiment it is possible to program a sequence of pulse pauses using, for example, a key pad or other selector. By way of example, in a 15 minute overall treatment the lamp may pulse on for 60 seconds and off for 60 seconds for the first 6 minutes and then pulse 5 seconds on and 5 seconds off for the rest of the treatment (9 minutes).

In another preferred embodiment the pulsing and pauses there between can be controlled by an external input, for example the surface temperature of the area being treated. For example the light could pulse until the nail had a surface temperature of 80 degrees F. and then could alternate pulses and pauses to maintain that temperature throughout treatment. Or the light could pulse until the patient pushed a button which would cause it to pause until the patient pushed the button a second time. Alternatively, the pulses could continue while the user's hand or foot was within the machine and pause while the user's hand or foot is removed.

In another preferred embodiment the device is similar to the first embodiment but is composed of two units, one which contains the power module and one which has the light. A variant of this embodiment is that one power unit can be used to power multiple light units.

In another preferred embodiment the device is similar to the first embodiment but is composed of multiple light units which can operate at the same time or independently. The lights may be enclosed in the light module or placed separate. Mirrors or other reflectors or reflective surfaces may be provide to direct light or to enhance the effect of the light. For example, an infrared light could come on to preheat the nail to the desired temperature and then could shut off or continue on when a second light providing UV is used.

In another preferred embodiment the device is similar to the first embodiment but has a means to detect if the light chamber is closed properly so that excessive light does not escape the chamber. One way this could be accomplished is with a photodetector that verifies low light levels in the chamber when the light is not on thus indicating light from the outside cannot enter (and thus light in the chamber cannot escape).

In another preferred embodiment the device is similar to the first embodiment but has means to measure the amount of light actually delivered to the area being treated. The results of the measurement may be used to alter the treatment, such as by changing the overall treatment time to apply the correct dosage, or to turn on, adjust or provide supplemental treatment means in addition to or in place of the main treatment light. For example, when using two lights, if the first light is not providing sufficient radiation a second light may be turned on. Or the number or duration of pulses may be increased in, regardless of the number of lights.

In a preferred embodiment this device could be used to treat skin cancer, warts, etc., with the area surrounding the cancer, warts, etc. masked off to allow the use of high dosages of light to inactivate and kill the cancerous cells, viruses causing warts, etc.

In a preferred embodiment, the area surrounding the area to be treated may be masked and the mask may remain in place for several treatments. The mask may have a medication that decreases any pain associated with the treatment either during or after the treatment and it may be left in place for a period of time as necessary.

In another preferred embodiment, the device is similar to the first embodiment but has an interlock to prevent the unit from operating if a mask is not present to shield the area not being treated. This can be done in a number of ways such as the mask having a special material that can be detected by the device such as a fluorescent dye in the mask, an RFID chip, or other ways that one skilled in the art would use.

In another preferred embodiment, the device is similar to the first embodiment but has an interlock to prevent the unit from operating if a mask is not present to shield the area not being treated.

In another preferred embodiment, the device is similar to the first embodiment but has interlocks to prevent the unit from operating if the unit is not correctly positioned.

In another preferred embodiment the device is similar to the first embodiment but uses air, water or other fluid for cooling for the power and/or lamp as they generate heat during treatment.

In another preferred embodiment the device is similar to the first embodiment but the area irradiated can be cooled by forced air, chilled air, cool water mist, or other means to cool the surface of the skin.

In another preferred embodiment the device is similar to the first embodiment but uses pneumatic skin flattening to enhance the treatment.

In another preferred embodiment the device is similar to the first embodiment but all energy is emitted in one large pulse of light.

In another preferred embodiment the device is similar to the first embodiment but all energy is emitted in several large pulses of light.

In another preferred embodiment the device is similar to the first embodiment but would use monochromatic UVC such as 254 nm emitted by a low pressure mercury lamp. In a variant of this embodiment the light would be emitted by an LED that emits at a germicidal wavelength.

In another preferred embodiment the device is similar to the first embodiment but the wide spectrum light would not be pulsed.

In another preferred embodiment the device is similar to the first embodiment but the pattern of pulsing would be altered to better treat an area.

In another preferred embodiment the device is similar to the first embodiment but would be capable of delivering 0.01 mj/cm² to 100 mj/cm².

In another preferred embodiment the device is similar to the first embodiment but would be capable of delivering between 100 mj/cm² and 100 J/cm².

In another preferred embodiment the device is similar to the first embodiment but the voltage pulse could be up to 1 gigavolt.

In another preferred embodiment the device is similar to the first embodiment but the current density per pulse could be up to 1 gigaamp.

In another preferred embodiment the device is similar to the first embodiment but the covering used for the extremity is specially designed to interface with the device. This would permit the covering to also block light from exiting the device. For example, the covering for treating fingernails could be similar to a glove with a square flange at the end near the wrist. The device would have a configuration permitting the square flange to be positioned so that it dovetailed with the device thus preventing stray light from exiting. The device could also be configured so that it would not operate unless such a flange was properly in place.

In another preferred embodiment the device is similar to the first embodiment but includes the provision of heat to the area treated from a supplemental source

In another preferred embodiment the device is similar to the first embodiment but includes more than one lamp to provide light to the area being treated.

In another preferred embodiment the device is similar to the first embodiment but would have a number of preprogrammed settings to deliver different doses based on different conditions.

In another preferred embodiment the device is similar to the first embodiment but would have the light (and gas mixture) and its envelope materials selected so that ozone would be generated to aide in treatment.

In another preferred embodiment the device is similar to the first embodiment but would have the light (and gas mixture) and its envelope materials selected so that ozone would not be generated as part of treatment.

In another further embodiment the device has a monochromatic and a wide spectrum light.

In another further embodiment the device is used in conjunction with antibiotics

In another further embodiment the device has a small number of discreet germicidal bandwidths such as 254 nm, 240 to 280 nm, etc.

In another further embodiment the light is delivered in one to 100,000 pulses, each having more than 10 mj of UVC

In another further embodiment the light is delivered in one to 100,000 pulses, each having more than 1 J of UV

In another further embodiment the light is delivered in one to 100,000 pulses, each having more than 10 J of combined UV, visible, and infrared light

The embodiments of this device may be incorporated into other device embodiments disclosed in this application or other devices that one skilled in the art could develop.

Preferred Embodiment for the Treatment of Nails in Combination with a Dryer for Nails and a UV Source for Affixing Artificial Nails

The preferred embodiments are descriptions of possible combinations of features and are illustrative, not exhaustive. One skilled in the art could develop similar combinations that include or exclude one or more certain items and these combinations are to be considered as part of this invention.

A preferred embodiment of the device can be used to prevent and treat nail infections and that can also be used to affix artificial nails or dry nail polish. This embodiment would be similar to any of the above mentioned devices that could treat nail infections with a modification that also permits the light and heat used to treat an infection to be used to dry nail polish or affix artificial nails. In the preferred embodiment a wide spectrum pulsed light could be used to treat an existing infection or provide a prophylaxis treatment. Once this was done the nails could be coated with polish and then device could be used to dry nails using light or heat, especially UV or infrared light or laser. In the preferred embodiment the light would switch to continuous mode to generate additional heat but less UVC. If instead of nail polish, the person would like to apply artificial nails the light could be used to catalyze the reaction of the gel to affix the nails. This would normally be a UV band but could be any band used as part of the process to affix nails. The light used to provide the UV or other bandwidth necessary to affix the nails could be provided by the light doing prophylaxis treatment or it could be a separate light. In a preferred embodiment, the device may recognize removal of the hand or nail and automatically progress from the treatment step to the drying step. Appropriate indicators, indicia or instructions could be presented on the device to instruct the user in the appropriate step under taken, such as “insert polished nails for drying,” etc. A mask used for prophylaxis treatment of the nails could remain in place for application of nail polish or artificial nails or could be removed after the initial prophylaxis treatment for infections.

In a preferred embodiment the light source for preventing or treating an infection would be the same as that used to dry the polish or catalyze a reaction to affix artificial nails. In this embodiment the light may be used in a different manner—for example pulsed in one mode and continuous in another. The timing and ultimate heat of operation may also vary for the different applications of the light. For example, the light may be held below a certain temperature during treatment (or prevention) for the comfort of the user by decreasing the pulses and increasing the pause times, and then allowing the temperature inside the device to increase as appropriate during drying applications. In an alternate embodiment different lights could be used in one or more combinations of preventing or treating an infection, drying nail polish, and catalyzing a reaction to affix artificial nails.

In another preferred embodiment the device would use light and heat to dry nail polish or affix artificial nails while at the same time delivering prophylaxis treatment to prevent infections. Although very little UVC light would penetrate below the nails in this mode of treatment due to the overlying polish or artificial nail the light would still disinfect all surfaces and the thermal energy would inactivate the small amount of organisms present before an infection was started. In the preferred embodiment the light would be highly pulsed and could quickly provide prophylaxis treatment and nail drying (or gel catalyzing) in a few minutes. This could be helpful in a nail salon setting to prevent the transfer of fungus between customers via foot baths or tools and equipment used by the salon.

In another preferred embodiment, the equipment used in manicures and pedicures could be sterilized by the light device. A special tray could be used to hold the equipment during sterilization before and/or after treatment of the user.

In another embodiment a UV light emitting primarily in the range of 320 nm to 400 nm would be used to catalyze the gel for artificial nails or dry polish.

### Preferred Embodiment for the Treatment of Skin

The preferred embodiments of a device to treat skin are descriptions of possible combinations of features and are illustrative, not exhaustive. One skilled in the art can develop similar combinations that include or exclude certain items and these combinations are contemplated as part of this invention.

A preferred embodiment of the device consists of a germicidal light that can irradiate the skin. A variety of germicidal light can be used including but not limited to UVC, pulsed light that is rich in UVC and contains other synergistic wavelengths, germicidal light that has been enhanced by using heat or other means to make it more effective, etc. Germicidal light can be monochromatic (such as low pressure mercury) or wide spectrum (such as xenon). Light can be supplied by a number of sources including low, medium, and high pressure mercury, xenon, deuterium lamp, LEDs (including organic LEDs), lasers, etc. These light sources could be used in combination with one another or alone. The light can be applied to the inside of a restricted areas by use of small lights that will fit inside the area to be treated or it may be generated outside the small area and directed into the area to be treated by a light guide or other means. Undesirable light can be minimized or eliminated by the use of filters, reflectors, and other means. For example, if it was desired to minimize or eliminate UVB to prevent sunburning, a notch filter that would eliminate or significantly decrease this bandwidth could be used.

A preferred embodiment of the device could be preprogrammed to deliver a fixed dose of light. It may have several such settings to cover a variety of situations. The settings could correspond to the dose ranges listed earlier in this application. The device may also be programmable for a custom dosage. The programming could also allow the light to turn off for set periods of time to prevent heat buildup in the unit or in the area being treated or for other reasons.

The preferred embodiment of the device would be portable and two such configurations are shown in FIGS. 11 and 12. The device would be completely contained in a unit sized to be held in a hand (FIG. 11) or it could have a separate unit that could be set on a table with a cable (light guide or power cable) extending to a hand unit (FIG. 12).

The completely contained handheld unit, FIG. 11, could have a pistol grip 200 with a trigger operator 202 that could serve as a safety switch. The device could have controls 204 that would permit the time, duration, and other parameters to be easily set. There could also be a string of LED lights 206 to indicate during operation how much time is left in the treatment (e.g., with lights turning off along the line of LEDs as the treatment proceeds). The device could have a power cord 208 or alternately the device could be rechargeable or run off batteries thus obviating the need for a power cord when in operation. The device could have a charging cradle for charging between uses. The device could use supercapacitors instead of batteries to provide for rapid recharging and high power in the handheld unit. The device could have a protective cap 210 when the unit is not in use. Alternately, the device could have a shutter that protects the unit when not in use. The unit could also be fitted with an iris attachment 212 or lens that allows the user to quickly vary the size of the area to be treated. The iris could be integral or could be an attachment that would fit over the unit similar to a lens cap.

The device with a separate base and treatment wand, FIG. 12, could have an integral docking bay 220 into the base unit 222 which could also house the power supply. The device could have controls 224 on the base unit that would permit the time, duration, and other parameters to be easily set or viewed, or such controls could be located on the treatment wand 226. There could also be indicators such as a string of LED lights 228 to indicate during operation how much time is left in the treatment (with lights turning off along the line of LEDs as the treatment proceeds) or to graphically indicate an upcoming pulsing pattern. The cable 230 connecting the two units could be a power cord with the light being located in the treatment wand or it could be a light guide with the light being generated in the base unit. The treatment wand could also be fitted with an iris attachment 212 or lens that allows the user to quickly vary the size of the area to be treated. The iris could be integral or could be an attachment that would fit over the unit similar to a lens cap.

In both the integrated handheld unit and the two piece base/treatment wand unit the controls for the device can varied from being very simple to complex. In one preferred embodiment the controls are very simple with only one setting. This control system would be very applicable for emergency treatment of an accidental needle prick or other small contaminated area. For many units a moderately complex control system might be desirable with several dedicated buttons for the most common treatments selected and the ability to custom program special settings. The control system could have a microprocessor capable of running a large number of programs. The control system could also have the ability to communicate with other device and computers by use of a cable connected to its programming/communications port or via a wireless interface. Data that can be exchanged may be programming for special treatments (data going from a computer to the unit) or details of treatment given (data going from the unit to a computer).

In a preferred embodiment this device could be used to treat skin cancer, warts, etc. with the area surrounding the cancer, warts, etc. masked off and high dosages of light used to inactivate and kill the cancerous cells, viruses causing warts, etc.

In a preferred embodiment the area surrounding the area to be treated may be masked and the mask may remain in place for several treatments. The mask may have a medication that decreases any pain associated with the treatment either during or after the treatment and it may be left in place for a period of time as necessary.

Most organisms that can cause a skin infection can be effectively inactivated with a dosage of approximately 1 to 20 mj/cm² of UVC. Wide spectrum pulsed light is several times more effective than UVC alone and if it is used to treat an area it is possible that a dose of wide spectrum light with approximately 5 mj/cm² of UVC would be sufficient. At a dosage of less than 20 mj/cm² erythema will be minimal and should not cause a problem. However, if erythema is a concern then a UVB filter can be applied over the lens to reduce erythema. The preferred embodiment of a light used to prevent infections in an area would use a pulsed wide spectrum light delivering a dosage of approximately 5 to 30 mj/cm² per treatment. Preferably the dosage would be delivered in 1 to 100 pulse delivered in one second or less. An alternative embodiment would be a monochromatic LED that delivered a dosage of approximately 5 to 30 mj/cm² of UVC.

Once an organism has caused an infection it is necessary to apply sufficient light to penetrate and inactivate the organisms. For very superficial infections the dosage may need to be increases by an order of magnitude to take into account that only about 10% of the light may penetrate thus a dosage of 50 to 300 mj/cm² of UVC may be required. For deeper infections the rate of penetration may be substantially less and in the range of 1% to less than 0.01% which may require a dosage of 500 mj/cm² to 3 J/cm² for 1% percent penetration to 50 to 300 J/cm² for a penetration rate of 0.01%. This dosage could be reduced significantly if pulsed wide spectrum light was used. For example the dosage for 0.01% penetration may be reduced to approximately 10 to 100 J/cm² of UVC or lower if pulsed wide spectrum light is used. If erythema is a concern then a UVB filter can be used to reduce erythema. The preferred embodiment of to treat infections would use a pulsed wide spectrum light delivering a dosage of approximately 500 mj/cm² to 10 J/cm² per treatment or more for severe infections.

The device could have any desired dosage, but is preferably capable of delivering doses as low as 1 mJ/cm² to as high as 100 J/cm² of UVC. More preferably the delivered dosage is between 10 mJ/cm² and 10 J/cm². Preferentially delivered using a broad spectrum light with a total light output of 12 to 4,800 J/cm².

The device could have a positive means to prevent light from being on when not irradiating an area to be treated.

The device could generate a small amount of ozone to help disinfection or can be screened out by proper glass selection.

The first preferred embodiment of the device to treat skin is a handheld, rechargeable device. The device could use rechargeable batteries. The device has a disposable tip. The disposable tip is designed to be applied on the skin for treatment. The device may have a positive means to ensure the device was correctly positioned and to prevent accidental exposure of the user's eyes to the light. The means may include an integral shield or a detector to determine if the device was in the correct location. The disposable tip is designed to diffuse the light evenly. The tip would be made of a material that was optically transparent to the germicidal light used. For example the tip could be TEFLON™ which if somewhat optically transparent to UVC (for example 2 mil FEP TEFLON transmits 90% of UVC). The preferred device would use pulsed light (one or more pulses) that was rich in UVC and that had other synergistic wavelengths. The device would have the capability of altering the pulses and also altering the time between pulses or sets of pulses. A preferred pulsing pattern would be 5 seconds of pulsing and 5 seconds of non-pulsing. The device would be capable of delivering between 1 mj/cm² to 10 J/cm² of UVC per treatment or its equivalent in another wavelength of germicidal light. Pulse widths can vary from 1 microsecond to 50 milliseconds are particularly contemplated as part of this invention as well as shorter pulses in the 1 to 1,000 femto, 1 to 1,000 pico, and 1 to 1,000 nanosecond range. A preferred variant of this pulse range is that the pulses produce light in the ultraviolet and visible range between 200 nm and 700 nm of which at least 1% is in the range of 200 nm to 280 nm and 10% is in the range of 200 nm to 400 nm. The device would have preprogrammed settings to permit controlled and uniform dosing. The device would have at least one preprogrammed setting to deliver between 1 mj/cm² to 10 J/cm² of UVC or would have other means to permit a desired dose to be given.

In another preferred embodiment the device is similar to the first embodiment but has a larger unit attached that provides power. In this preferred embodiment the light is part of a handheld unit.

In another preferred embodiment the device is similar to the first embodiment but has a larger unit attached that provides both power and generates the light. The light is then directed to the treatment area via a light guide or other similar means.

In another preferred embodiment the device is similar to the first embodiment but does not use a disposable tip but instead uses a disposable cover for the tip.

In another preferred embodiment the device is similar to the first embodiment but does not use a disposable tip. Instead the tip is easily sterilized for reuse.

In another preferred embodiment the device is similar to the first embodiment but uses removable batteries, removable rechargeable batteries or a removable battery pack so that the unit could be used for a considerable time by swapping out batteries.

In another preferred embodiment the device is similar to the first embodiment but uses supercapacitors to permit quick recharging and high power densities.

In another preferred embodiment the device is similar to the first embodiment but the tip could be made of a composite material such as fused quartz to provide strength coated with Teflon to prevent breakage. In a variant of this embodiment the tip could have a permanent tip made of fused quartz or sapphire or other optically transparent material and a disposable cover made of Teflon or other optically transparent thermoplastic.

In another preferred embodiment the device is similar to the first embodiment but the tip could be coated with a compound that would filter out undesirable wavelengths such as UVB.

In another preferred embodiment the device is similar to the first embodiment but would use monochromatic UVC such as 254 nm emitted by a low pressure mercury lamp. In a variant of this embodiment the light would be emitted by an LED that emits at a germicidal wavelength.

In another preferred embodiment the device is similar to the first embodiment but the wide spectrum light would not be pulsed.

In another preferred embodiment the device is similar to the first embodiment but the pattern of pulsing would be altered to better treat an area.

In another preferred embodiment the device is similar to the first embodiment but would be capable of delivering 0.1 mj/cm² to 100 mj/cm².

In another preferred embodiment the device is similar to the first embodiment but would be capable of delivering between 100 mj/cm² and 100 J/cm².

In another preferred embodiment the device is similar to the first embodiment but would have a number of preprogrammed settings to deliver different doses based on different conditions.

In another preferred embodiment the device is similar to the first embodiment but would have the light (and gas mixture) and its envelope materials selected so that ozone would be generated to aide in treatment.

In another preferred embodiment the device is similar to the first embodiment but would have the light (and gas mixture) and its envelope materials selected so that ozone would not be generated to aide in treatment.

In another further embodiment the device has different models and tips to permit a wide range of treatment.

In another further embodiment the device has a multiplicity of lights

In another further embodiment the device has a monochromatic and a wide spectrum light.

In another further embodiment the device is used in conjunction with antibiotics

In another further embodiment the device is used in conjunction with replacement of inactivated organism with a non-pathogenic culture.

In another further embodiment the device has a small number of discreet germicidal bandwidths such as 254 nm, 240 to 280 nm, etc.

The device can have programmable pulsing and ‘resting’ can be set to deliver optimal light and heat while not damaging the area being treated.

The device could be used in conjunction with goggles to prevent light from hurting eyes.

The device could be used in conjunction with a shield interposed between the area being treated and the eyes of the subject to prevent light from harming the eyes.

The embodiments of this device may be incorporated into other device embodiments disclosed in this application or other devices that one skilled in the art could develop.

### Preferred Embodiment for the Treatment of Leishmania

The preferred embodiments are descriptions of possible combinations of features and are illustrative, not exhaustive. One skilled in the art could develop similar combinations that include or exclude certain items and these combinations are contemplated as part of this invention.

A variant of the device to treat skin conditions is proposed as containing modifications to treat leishmaniasis and other infections, diseases, and disorders that respond well to a combination of germicidal light and thermal energy. The germicidal light and thermal energy in a preferred embodiment consists of a combination of two bandwidths—one which can damage the organism causing the infection and one which provides heat to work synergistically with the light which damages the organism or which stimulates the body's natural defenses. One aspect of this unique invention is that the two bandwidths combined act far more effectively than the sum of each of the wavelengths acting individually.

FIG. 13 illustrates a covering 250 can be used to screen or filter out UV 252 in the area not being treated. The covering could be relatively opaque to UV but relatively transparent to much of the infrared spectrum 254 so that the surrounding area could receive significant infrared to raise the skin's temperature. This could stimulate blood flow and the body's natural defenses as well as therapeutically stressing any undesirable organisms. Alternatively the covering could also block out a substantial amount of infrared in addition to UV.

A variety of germicidal light can be used including but not limited to UVC, pulsed light that is rich in UVC and contains synergistic wavelengths, germicidal light that has been enhanced by using heat or other means to make it more effective, etc. Germicidal light can be monochromatic (such as low pressure mercury or a corona discharge excimer) or wide spectrum (such as xenon). Light can be supplied by a number of sources including low, medium, and high pressure mercury, xenon, deuterium lamp, LEDs (including organic LEDs), corona discharge excimer, lasers, etc. These light sources could be used in combination with one another or alone or in sequence. FIG. 14 illustrates a device with a UVC light 260 and an infrared light 262 both of which use parabolic reflectors 264 to direct light to the treated area. Undesirable light can be minimized or eliminated by the use of filters, reflectors, and other means. For example, if it was desired to minimize or eliminate UVB to prevent sunburning, a notch filter that would eliminate or significantly decrease this bandwidth could be used. FIG. 15 illustrates the use of a notch UVB filter 270 with a wide spectrum lamp 272. Comparison of the spectrum before use of the notch filter 274 and after the use of the notch filter 276 shows that UVB has been screened.

To treat leishmania it is necessary to apply sufficient light to penetrate and inactivate the organisms. For very superficial infections the dosage may be approximately 50 to 300 mj/cm² of UVC. For deeper infections the rate of penetration may be substantially less and in the range of 1% to less than 0.01% which may require a dosage of 500 mj/cm² to 3 J/cm² for 1% percent penetration to 50 to 300 J/cm² for a penetration rate of 0.01%. This dosage could be reduced significantly if pulsed wide spectrum light was used. For example the dosage for 0.01% penetration may be reduced to approximately 10 to 100 J/cm² of UVC or lower if pulsed wide spectrum light is used. If erythema is a concern then a UVB filter can be used to reduce erythema. The preferred embodiment of to treat leishmania would use a pulsed wide spectrum light delivering a dosage of approximately 500 mj/cm² to 10 J/cm² per treatment or more for severe infections.

The device could be preprogrammed to deliver a fixed dose of light. It may have several such settings to cover a variety of situations. It may also be programmable for a custom dosage. The programming could also cover if the light should turn off for set periods of time to control the heat generated in order to enhance treatment. The device could be portable. It could be completely contained in a unit sized to be held in a hand or it could have a separate unit that could be set on a table with a cable (light guide or power cable) extending to a hand unit. The device could be rechargeable or run off replaceable batteries, thus obviating the need for a power cord when in operation. The device could have a charging cradle to charge it between uses. The device could use supercapacitors instead of batteries to provide for rapid recharging and high power in the handheld unit.

The device could have a dosage as low as 10 mJ/cm² to as high as 300 J/cm² of UVC. It could have overall light dosages in the range of 100 mj/cm² to 10,000 J/cm². Preferred dosage is between 50 mJ/cm² and 100 J/cm² of UVC. Preferentially delivered using a broad spectrum light which has a filter to remove most of the UVB generated. Additionally, the device would have the capability of applying heat to raise the temperature of the area being treated for the desired amount of time.

The ratio of UVC to heat applied can be varied by changing several parameters including the magnitude of pulsing, the length of each pulse, the pulse rate, and pauses in the pulsing each which will be discussed in the following paragraphs.

Pulsing of lamps such as those filled with xenon gas shifts the spectrum of the emitted light towards the shorter wavelengths. The higher the magnitude of the pulse, the higher the percentage of short wavelength light. This can be illustrated by comparison of two modes of pulsing in xenon flash lamps which are termed low current and high current pulsing. Representative graphs of these two types of pulsing are shown in FIG. 16. Thus a practitioner skilled in the art can vary the magnitude of the pulses to obtain the desire mix of longer and shorter wavelengths. Additionally, several lamps can be used together, each with different magnitude of pulses to provide more flexibility in obtaining the desired mix of wavelengths.

Another parameter that can be varied is the length of the pulse. Normal pulsed xenon lamps have pulse durations of 10 milliseconds to 50 milliseconds and this rate can be varied to achieve the desired penetration profile through the area being treated. However, even shorter pulses measured in nanoseconds (or even picoseconds) may also yield desirable penetration profiles. There is evidence with ultrafast lasers and with pulsed electrical fields that such rapid pulsing may overcome the defenses of an organism and critically damage it. Therefore, control of the pulsing of the light (including very rapid nanosecond and picoseconds) is also contemplated as part of the device to treat leishmaniasis and other infections.

The pulse rate can also be varied to achieve a desire penetration profile for the light being applied. Very rapid pulses build up heat without providing a chance for it to dissipate. This can be appropriate if the buildup of heat will increase this efficacy of the treatment. However, excessive heat buildup could damage the skin or other part of the body being treated and therefore the correct selection of pulse rate is an important variable in delivering treatment.

Another parameter that can be varied to achieve the desired treatment is the insertion of pauses in the treatment. Pausing treatment for a time permits heat to be dissipated. It also allows for other adjunct treatment to be provided such as blowing of air on the area being treated or application of misted cool water (though these can also be done during pulsing). It is not only the percent of time a treatment is paused but also the length of each pulsing/pause cycle that is important. For example trials on treating onychomycosis have indicated that though a total pulse time of 10 minutes and a total rest time of 10 minutes (20 minutes total treatment) provides acceptable treatment, the pulse/pause time is very important in determining effectiveness and user comfort. For example, if a pulse time of 1 minute on and a rest time of 1 minute off is used then a subject may begin to experience intense discomfort after only half of the total treatment time has elapsed. If the pulse/pause rate is reduced to 10 sec on/10 sec off then perhaps two-thirds of a 20 minute total treatment can be given before intense discomfort sets in. However, if the pulse rate is set to 5 sec on/5 sec off there is enough heat dissipation to permit the entire 20 minute treatment to be given. It may also be desirable to vary the pulse/pause times as treatment progresses. For example, it may be possible to start with a 60 sec on/60 sec off pulse/pause rate and then decrease it in 5 second increments after several times (i.e. this pulse/pause rate could be given 5 times and then reduced to 55 sec on/55 sec off for the next 5 rounds, and then 50/50 seconds, etc.). This can be seen illustrated in FIG. 17. Another variation that can enhance treatment is that the temperature of the area being treated can be monitored and the treatment could automatically vary to maintain a desired temperature for a set amount of time. For example, the treatment could be pulsed continuously until a surface temperature of 50 C was obtained and then the light could be pulsed on/off in a 5 sec on/5 sec off cycle as often as necessary to maintain this temperature for a total of 1 minute of treatment at 50 C. Once treatment is finished adjunct treatment such as application of cool air or cool water mist could be applied to minimize any damage caused by heat.

It should be noted that while the UVC portion of the light does not penetrate as deeply as some other wavelengths, it is extremely effective at low doses and thus can have a impact at dosage levels much smaller than other wavelengths. In addition, the UVC is extremely effective at sterilizing the surface of the infection thus significantly reducing the chance of outside contamination.

The preferred embodiments are descriptions of possible combinations of features and are illustrative, not exhaustive. One skilled in the art could develop similar combinations that include or exclude certain items and these combinations are contemplated as part of this invention.

The preferred device to treat leishmania would use pulsed light (one or more pulses) that was rich in UVC and that had other synergistic wavelengths and would screen out the majority of UVB. The device would have the capability of altering the pulses and also altering the time between pulses or sets of pulses in order to generate and sustain heating of an area for a specified amount of time. A preferred pulsing pattern would quickly generate and sustain heat to the area being treated that was sufficient to inactivate the organism causing leishmaniasis and/or to stimulate the body's natural healing defenses. One preferred pattern of heating would be to quickly raise the area being treated to a surface temperature of 50 C and maintain that temperature for 30 to 45 seconds. A preferred embodiment would have a means of measuring temperature of the skin to precisely control the temperature. This means could be an RTD, thermocouple sensor, infrared thermography or other temperature measuring device. The temperature measuring device could be use by the operator to manually maintain temperature or it could be interlocked with the device and automatically produce and maintain a set temperature for a set period of time.

A variant of the above preferred embodiment would be similar to above but the area would be quickly heated to maintain a temperature of between 45 C and 50 C for approximately one to two minutes. An additional variant would use a masking system that permitted some of the applied heat to penetrate the area being masked while still blocking any UV applied. The longer heating period and the larger area of heating will permit the effect of the treatment to penetrate more deeply thus stressing the organisms causing the infection and improving the efficacy of the treatment. Heat can be applied by a number of means including wide spectrum light, infrared light, radio waves, microwave, hot air, etc.

In another preferred embodiment, the device is similar to the first embodiment but has an interlock to prevent the unit from operating if a mask is not present to shield the area not being treated.

A preferred embodiment would make use of a mask around the area being treated with the mask being made of a material that was opaque to UV but relatively transparent to infrared light. In this preferred embodiment the area being treated would also have its temperature rise in order to stimulate the body's natural defenses. A variant on the preferred embodiment would be to have an ointment applied afterward to minimize the aftereffects of applying heat. A variant of the ointment could be the use of an ointment that would kill protozoans.

The device would be capable of delivering between 10 mj/cm² to 100 J/cm² of UVC per treatment or its equivalent in another wavelength of germicidal light. The device would have preprogrammed settings to permit controlled and uniform dosing. The device would have at least one preprogrammed setting to deliver between 10 mj/cm² to 100 J/cm² of UVC or would have other means to permit a desired dose to be given.

The device may use precalculated dosages according to the methods outlined earlier in this application.

The device would also be capable of delivering sufficient visible or infrared light to raise the temperature of the skin to a level which would adversely affect the leishmania organisms and/or stimulate the body's natural defenses.

In another preferred embodiment the device is similar to the first embodiment but has a means to prevent temperature buildup on the skin surface by means such as application of cooling water or air or substance having similar cooling ability. This would permit the application of deep heat while minimizing damage to the surface of the skin.

In another preferred embodiment the device is similar to the first embodiment but has separate sources to generate UVC and heat. In a variant of this preferred embodiment the heat can be generated by a light such as an infrared or visible light or it can be generated by other means such as the application of hot air, radio waves, microwaves, wide spectrum light or another substance.

In another preferred embodiment the device is similar to the first embodiment but has a larger unit attached that provides both power and generates the light. The light is then directed to the treatment area via a light guide or other similar means.

In another preferred embodiment the device is similar to the first embodiment but uses removable batteries so that the unit could be used for a considerable time by swapping out batteries.

In another preferred embodiment the device is similar to the first embodiment but uses supercapacitors to permit quick recharging and high power densities.

In another preferred embodiment the device is similar to the first embodiment but would use monochromatic UVC such as 254 nm emitted by a low pressure mercury lamp or a corona discharge excimer lamp and a second source to provide heat to the area. In a variant of this embodiment the light would be emitted by an LED that emits at a germicidal wavelength and another that emits in the infrared range.

In another preferred embodiment the device is similar to the first embodiment but the treatment light can be placed directly on the area being treated to enhance efficacy.

In another preferred embodiment the device is similar to the first embodiment but the wide spectrum light would not be pulsed.

In another preferred embodiment the device is similar to the first embodiment but the pattern of pulsing would be altered to better treat an area.

In another preferred embodiment the device is similar to the first embodiment but would be capable of delivering 0.1 mj/cm² to 100 mj/cm² of UVC.

In another preferred embodiment the device is similar to the first embodiment but would be capable of delivering between 1 mj/cm² and 10 J/cm² of UVC.

In another preferred embodiment the device is similar to the first embodiment but would have a number of preprogrammed settings to deliver different doses based on different conditions.

In another preferred embodiment the device is similar to the first embodiment but would have the light (and gas mixture) and its envelope materials selected so that ozone would be generated to aide in treatment.

In another preferred embodiment the device is similar to the first embodiment but would have the light (and gas mixture) and its envelope materials selected so that ozone would not be generated to aid in treatment.

In another preferred embodiment the initial treatment would be followed by Skin Contact Monochromatic Infrared Energy Therapy (MIRE) to enhance efficacy.

In another preferred embodiment the environment of infected area would be modified to stress the organism and/or mobilize the body's natural defenses. Modifications can include use of ultrasound, heat, change of oxygen content, change of pH, change of temperature, etc. For example, after treatment the area could be covered with a material that was oxygen impervious since oxygen is needed for Leishmania infantum promastigotes to thrive. Thus, depriving them of one source of oxygen could greatly hinder their viability.

The embodiments of this device may be incorporated into other device embodiments disclosed in this application or other devices that one skilled in the art could develop.

### Preferred Embodiment for Wounds, Acne, Psoriasis, and Other Infections and Disorders

The preferred embodiments are descriptions of possible combinations of features and are illustrative, not exhaustive. One skilled in the art could develop similar combinations that include or exclude certain items and these combinations are contemplated as part of this invention.

A preferred embodiment of a device to treat skin disorders such as acne and psoriasis may use a configuration that permits precise application of germicidal light to the areas to be treated. For example, in order to treat a face with acne one could use a series of lights configured to provide uniform light over the entire area of the mask but which was also controllable so that certain areas could be selectively dimmed or turned off if the specific area required little or no treatment. FIG. 18 shows one configuration of this mask with a series of small xenon flash lamps 280 set in a matrix of a face mask. A practitioner could take a digital photo of the face and then identify the areas to be treated on a computer screen that showed the digital image of the face using a pen on a touch sensitive screen which then configures which lights to turn on and for what strength and duration they should be turned on. Alternately there may be one or more lights that illuminate uniformly and an LCD shutter matrix or similar light gate could open or close areas so that light could pass through to irradiate the areas to be treated. Other variants include the use of small LEDs or other lights and the mixture of one or more of several kinds of light such as small UVC and IR LEDs. One variant of this embodiment could use a mask that had a thin layer of Teflon or other optically transparent material over the light so that it can be easily cleaned. Alternatively, there could be a mask made of disposable optically transparent material that could be inserted after each use, or having selectively transparent and nontransparent areas. Another variant of this embodiment could use a digital photograph to serve as a template to cut out a mask (or build a mask having appropriate transparent areas) that fits the area to be treated. The computer could then send the cutout image to a printer to have it printed on a mask or there could be a custom mask cutter attached to the computer.

FIG. 19 shows another preferred embodiment where the lights 300 are contained in a relatively flexible array 302 similar to a bandage or cloth and that can be applied over wounds and burns for treatment. The flexible array may have an easily cleanable optically transparent cover or it may use a disposable optically transparent cover or it may not require either if it does not come in contact with the area being treated. In one variant of this preferred embodiment the light matrix could be configured to only activate the lights to treat a specific configuration. For example, an oval size psoriasis plaque 304 that is on the arm and is 2-inches by 3-inches could be covered by a 6-in square flexible array which would only activate the lights where the psoriasis was 306. Additionally, the strength of the treatment could be less on the edges of the plaque and stronger in the middle. The configuration of the lights to be activated could be based on a digital image such as described for acne treatment earlier or it could use methods such as detecting an outline traced around the area being treated 308 using a special marking pen or the matrix detecting which area is raised or has a different coloration or temperature or other attribute peculiar to the area requiring treatment that can be used as a guide on where to apply light. In a variant of this embodiment a mask that is larger than the flexible array can be applied with only the affected area being exposed for treatment. For example, if a 6-in square array was used the mask could be 8-in square with the appropriate area removed to expose the disorder to be treated.

In another preferred embodiment, the device is similar to the first embodiment but has an interlock to prevent the unit from operating if a mask is not present to shield the area not being treated.

In the preferred embodiment dosage ranges to treat wounds, acne, psoriasis, and other disorders would be approximately the same as for treatment of other skin diseases and disorders and could range from 10 mj/cm² to up to 300 J/cm² of UVC. Use of pulsed wide spectrum light can significantly decrease the dosage required.

In a preferred embodiment germicidal light can be applied as part of an enriched oxygen or hyperbaric treatment. The germicidal light can be of the type that also generates ozone and singlet oxygen to help enhance treatment and which will be produced in higher than normal concentrations of oxygen.

The embodiments of this device may be incorporated into other device embodiments disclosed in this application or other devices that one skilled in the art could develop.

Preferred Embodiment Incorporated into a Glucose Monitor or Other Device

The preferred embodiments are descriptions of possible combinations of features and are illustrative, not exhaustive. One skilled in the art could develop similar combinations that include or exclude certain items and these combinations are contemplated as part of this invention.

A preferred embodiment of the device could be used to prepare and to post-treat areas that will be punctured by a needle to prevent infections. The device might be incorporated into the needle apparatus to provide irradiation before, during, or after the needle was inserted and withdrawn or any combination thereof.

Additionally, an embodiment of this invention treatment could be used to treat other areas such a preparing an area that will punctured by a needle. The treatment could also be used after the area has been penetrated to prevent infection. This would be especially useful for diabetics who have to repeatedly take blood samples to monitor their blood sugar levels.

FIG. 20A shows another preferred embodiment the device that is similar to the first embodiment but the tip is designed to provide light to a fixed area such that immediately around a site being punctured by a needle. In a variant of this embodiment the tip is designed to treat an area approximately 1-in in diameter. In a variant of this embodiment the tip is cylindrical and extends from the light emitting area to the area being treated. The tip can be used to isolate the area being treated from its surroundings and thus exposing only the treated area to the light emitted. In a variant of this embodiment the device has a photodetector inside the confined area that determines if the light is low enough to indicate that the tip is in contact with the area to be treated thus forming a seal from other areas and making the area to be treated dark. The photo detector is interlocked with the light so that it can only operate if the photodetector detects a low level of light. In a variant of this embodiment the device has an infrared detector inside the confined area that determines if the infrared signature is high enough to indicate that the tip is in contact with the area to be treated. The infrared detector is interlocked with the light so that it can initially operated only if the infrared detector has the required reading. In a variant of this embodiment the device has both a photodetector and an infrared detector that are used to make sure the device is properly positioned. In another variant of this embodiment one or more means are used to ensure that the detector is properly positioned. Other detectors are also contemplated and could be used, such as a pressure detector for detecting when the device has reached a stop, such as a narrowing in the upper nasal cavity.

FIG. 20B shows another preferred embodiment with a device is similar to first embodiment but the light is used to disinfect a site penetrated by a needle or other similar apparatus, for example a lancet to take a blood sample for testing of blood glucose. In this embodiment the light may be built into the device that has the needle. In a variant of this embodiment the light may be located on the instrument such that light can be applied before, during, and/or after the needle penetrates. In another variant, the light may be of low enough intensity and for such a short period of time that it has a very low probability of damaging the eyes and so does not require a shield around it. In another variant the shield may be a means to direct the light to the area being treated, but need not be in contact with the area being treated. In another variant the device may not need a tip as such if there are other means to direct the light to the area being treated. In another variant the light may be interlocked with the act of taking a blood sample and trigger when the sample is taken and/or immediately thereafter. In another variant the light may be separate from the needle or other apparatus that penetrates the skin or it may be located on a different part of the device. For example, the light may be on the other side of the testing device and to use it the device would need to be rotated and the light positioned over the area to be treated (i.e. the area the needle penetrated). In a variant of this embodiment this type of light may be used to irradiate an accidental needle stick to reduce or eliminate the chance for infection. In this latter variant the dosage delivered may be much higher than normal in order to provide maximum efficacy against infection.

Most organisms that can cause a skin infection can be effectively inactivated with a dosage of approximately 1 to 20 mj/cm² of UVC. Wide spectrum pulsed light is several times more effective than UVC alone and if it is used to treat an area it is possible that a dose of wide spectrum light with approximately 5 mj/cm² of UVC would be sufficient. At a dosage of less than 20 mj/cm² erythema will be minimal and should not cause a problem. However, if erythema is a concern then a UVB filter can be applied over the lens to reduce erythema. The preferred embodiment of a light used to prevent infections in an area that has a puncture wound would use a pulsed wide spectrum light delivering a dosage of approximately 5 to 30 mj/cm² per treatment. Preferably the dosage would be delivered in 1 to 100 pulse delivered in one second or less. An alternative embodiment would be a monochromatic LED that delivered a dosage of approximately 5 to 30 mj/cm² of UVC.

In a preferred embodiment a dosage of 5 mJ/cm² to 500 mj/cm² of UVC would be applied per use. For the emergency variant to sterilize an area that could be contaminated the dosage may extend up to 10 J/cm² of UVC. Preferably the light would be highly directional (i.e. focused specifically on the area being treated) and delivered quickly (in less than 1 second for normal use and 5 seconds for emergency use if a high dosage is required).

The embodiments of this device may be incorporated into other device embodiments disclosed in this application or other devices that one skilled in the art could develop.

### Preferred Embodiment for the Treatment of Nostrils and Nasal Passages

The preferred embodiments of a device to treat skin are descriptions of possible combinations of features and are illustrative, not exhaustive. One skilled in the art could develop similar combinations that include or exclude certain items and these combinations are claimed as part of this invention.

A preferred embodiment of the device could be used to treat or prevent nasal infections. The device could also be used to reduce the microbial load of a nasal passageway. The device could also be used effectively against MRSA to reduce or eliminate it. The device could also be used in conjunction with antibiotics to increase its effectiveness.

FIG. 21 shows a preferred embodiment of the device to treat nostrils

The preferred embodiment can use a variety of germicidal light can be used including but not limited to UVC, pulsed light that is rich in UVC and contains synergistic wavelengths, germicidal light that has been enhanced by using heat or other means to make it more effective, etc. Germicidal light can be monochromatic (such as low pressure mercury) or wide spectrum (such as xenon). Light can be supplied by a number of sources including low, medium, and high pressure mercury, xenon, plasma lamps, surface discharge lamps, deuterium lamp, LEDs (including organic LEDs), lasers, etc. These light sources could be used in combination with one another or alone. The light can be applied to the inside of a restricted areas such as the nasal passageways by use of small lights that will fit inside the nasal cavity or it may be generated outside the small area and directed into the area to be treated by a light guide or other means. Attachments could also be used to irradiate other nasal passages. Undesirable light can be minimized or eliminated by the use of filters, reflectors, and other means. For example, if it was desired to minimize or eliminate UVB to prevent sunburning, a notch filter that would eliminate or significantly decrease this bandwidth could be used.

The device could use a special tip that can be disposable. Alternatively the tip could have a disposable cover on it. Another alternative is that the tip could be easily sterilized between uses. The tip could also direct or focus the light as desired. Alternately, the tip could be used to evenly diffuse the light. The tip could be coated or fabricated so that it filtered out undesirable wavelengths. The tip could be fitted over fiber optic tip or other light source. The tip could be interlocked with light so that light cannot be triggered until tip is on properly (keeps light from getting in eyes). In a preferred embodiment the tip would be less than approximately ½ inch in diameter and approximately ½ to 2-inches long. In a preferred embodiment the tip may have longitudinal folds to increase its surface area and to help it diffuse or deliver light to the nasal cavity better. In a variant embodiment the tip may move longitudinally or rotate to better disperse light in the nasal cavity.

The device could be preprogrammed to deliver a fixed dose of light. It may have several such settings to cover a variety of situations. It may also be programmable for a custom dosage. The programming could also cover if the light should turn off for set periods of time to prevent heat buildup in the unit or in the area being treated or for other reasons. The device may use preprogrammed dosages similar to the dosage tables provided in this application.

The device could be portable. It could be completely contained in a unit sized to be held in a hand or it could have a separate unit that could be set on a table with a cable (light guide or power cable) extending to a hand unit. The device could be rechargeable or run off batteries thus obviating the need for a power cord when in operation. The device could have a charging cradle to charge it between uses. The device could use supercapacitors instead of batteries to provide for rapid recharging and high power in the handheld unit.

Most organisms that can are in the nasal cavity can be effectively inactivated with a dosage of approximately 1 to 20 mj/cm² of UVC. Wide spectrum pulsed light is several times more effective than UVC alone and if it is used to treat an area it is possible that a dose of wide spectrum light with approximately 5 mj/cm² of UVC would be sufficient. However, with the nares blocking a substantial amount of light it may be necessary to increase the dosage by a factor of 10 to 100. It should be noted though that it is not necessary to totally eradicate organisms in the nasal cavity especially if the area will be subject to adjunct recolonization. Therefore, a dosage of approximately 10 times, or 10 to 200 mj/cm² of UVC or approximately 50 mj/cm² of UVC provided using wide spectrum pulsed light may be most appropriate. If erythema is a concern then a UVB filter can be used to reduce erythema. The preferred embodiment of a light used to reduce organisms in the nasal cavity would use a pulsed wide spectrum light delivering a dosage of approximately 20 to 100 mj/cm² per treatment. Preferably the dosage would be delivered in 1 to 100 pulses delivered in one second or less. An alternative embodiment would be a monochromatic LED that delivered a dosage of approximately 20 to 300 mj/cm² of UVC.

Dosages used to prevent and treat infections of the nasal cavity or inactivate mast cells to reduce allergies would be similar to those used to prevent and treat skin infections.

The device could have any desired dosage, but is preferably capable of delivering doses as low as 10 mJ/cm² to as high as 300 J/cm² of UVC. More preferably the delivered dosage is between 50 mJ/cm² and 10 J/cm² of UVC. Preferentially delivered using a broad spectrum light with a total dosage between 12 j/cm² and 4,800 j/cm².

The device could have a positive means to prevent light from being on when not irradiating a nasal passageway

In a preferred embodiment this device could be used to treat tumors and polyps with the area surrounding masked off and high dosages of light used to inactivate and kill the cancerous cells.

In a preferred embodiment the area surrounding the area to be treated may be masked and the mask may remain in place for several treatments. The mask may have a medication that decreases any pain associated with the treatment either during or after the treatment and it may be left in place for a period of time as necessary.

The device could be used for the treatment of other sinus conditions such as allergies and could also be used to inactivate mast cells.

The device could be used in conjunction with goggles to prevent light from hurting eyes.

The first preferred embodiment is a handheld, rechargeable device. The device would use rechargeable batteries. The device has a disposable tip. The disposable tip is designed to be inserted into a nostril for treatment. The device may have a positive means to ensure the device was inserted correctly and would not flash light to the eyes. The means might be an integral shield or a detector to determine if the device was in the correct location. The disposable tip is designed to diffuse the light evenly. The tip would be made of a material that was optically transparent to the germicidal light used. For example the tip could be TEFLON™ which if somewhat optically transparent to UVC (for example 2 mil FEP TEFLON transmits 90% of UVC). The preferred device would use pulsed light (one or more pulses) that were rich in UVC and that had other synergistic wavelengths. The device would have the capability of altering the pulses and also altering the time between pulses or sets of pulses. A preferred pulsing pattern would be 5 seconds of pulsing and 5 seconds of non-pulsing. The device would be capable of delivering between 1 mj/cm² to 10 J/cm² of UVC per treatment or its equivalent in another wavelength of germicidal light. The device would have preprogrammed settings to permit controlled and uniform dosing. The device would have at least one preprogrammed setting to deliver between 1 mj/cm² to 10 J/cm² of UVC or would have other means to permit a desired dose to be given.

In another preferred embodiment the device is similar to the first embodiment but has a larger unit attached that provides power. In this preferred embodiment the light is part of a handheld unit.

In another preferred embodiment the device is similar to the first embodiment but may have a larger unit attached that provides both power and generates the light. The light is then directed to the treatment area via a light guide or other similar means.

In another preferred embodiment the device is similar to the first embodiment but does not use a disposable tip but instead uses a disposable cover for the tip.

In another preferred embodiment the device is similar to the first embodiment but the tip that is inserted in a nostril has a multiplicity of fiber optics tips to diffuse light around the nares of the nostril. The multiplicity of fiber optics could be disposable or reusable. In a variant, other means could be used to diffuse the light around the nares such as a reflective or diffusive gel, etc. The gel could be applied first or it could be on the tip of the device.

In another preferred embodiment the device is similar to the first embodiment but does not use a disposable tip. Instead the tip is easily sterilized for reuse.

In another preferred embodiment the device is similar to the first embodiment but uses removable batteries, removable rechargeable batteries or a removable battery pack so that the unit could be used for a considerable time by swapping out batteries.

In another preferred embodiment the device is similar to the first embodiment but uses supercapacitors to permit quick recharging and high power densities.

In another preferred embodiment the device is similar to the first embodiment but the tip would be used to direct the light instead of diffuse it.

In another preferred embodiment the device is similar to the first embodiment but the tip could be made of a composite material such as fused quartz to provide strength coated with Teflon to prevent breakage. In a variant of this embodiment the tip could have a permanent tip made of fused quartz or sapphire or other optically transparent material and a disposable cover made of Teflon or other optically transparent thermoplastic.

In another preferred embodiment the device is similar to the first embodiment but the tip could be coated with a compound that would filter out undesirable wavelengths such as UVB.

In another preferred embodiment the device is similar to the first embodiment but would use monochromatic UVC such as 254 nm emitted by a low pressure mercury lamp. In a variant of this embodiment the light would be emitted by an LED that emits at a germicidal wavelength.

In another preferred embodiment the device is similar to the first embodiment but the wide spectrum light would not be pulsed.

In another preferred embodiment the device is similar to the first embodiment but the pattern of pulsing would be altered to better treat an area.

In another preferred embodiment the device is similar to the first embodiment but would have a number of preprogrammed settings to deliver different doses based on different conditions. In this embodiment the dosages used could be derived from the dosage charts in this application.

In another preferred embodiment the device is similar to the first embodiment but would have the light (and gas mixture) and its envelope materials selected so that ozone would be generated to aide in treatment.

In another preferred embodiment the device is similar to the first embodiment but would have the light (and gas mixture) and its envelope materials selected so that ozone would not be generated to aide in treatment.

In another further embodiment the device has different models and tips to permit a wide range of treatment.

In another further embodiment the device has a multiplicity of lights

In another further embodiment the device has a monochromatic and a wide spectrum light.

In another further embodiment the device is used in conjunction with antibiotics

In another further embodiment the device is used in conjunction with replacement of inactivated organism with a non-pathogenic culture.

In another further embodiment the device has a small number of discreet germicidal bandwidths such as 254 nm, 240 to 280 nm, etc.

In another further embodiment the device has the preferred dose could be set at the level determined to inactivate mast cells which cause an allergic reaction.

The device can have programmable pulsing and ‘resting’ can be set to deliver optimal light and heat while not damaging nasal passageway or other area.

The device can be used as a prophylactic to lower the amount of microbes in the nose. It can be used in addition with antibiotics (including phages). Can be used in addition to the addition of non-pathogenic organisms to first decrease the native organisms and then recolonize with harmless organisms.

The device could be used in conjunction with goggles to prevent light from hurting a user's or patient's eyes.

The device could be used in conjunction with a shield interposed between the area being treated and the eyes of the subject to prevent light from harming the eyes.

The device can generate a small amount of ozone to help disinfection or can be screened out by proper glass selection.

The device is specifically contemplated as a prophylactic treatment for MRSA in nose.

The embodiments of this device may be incorporated into other device embodiments disclosed in this application or other devices that one skilled in the art could develop.

### Preferred Embodiment for Treatment of the Mouth

The preferred embodiments are descriptions of possible combinations of features and are illustrative, not exhaustive. One skilled in the art could develop similar combinations that include or exclude certain items and these combinations are contemplated as part of this invention.

A preferred embodiment of the invention is a device that can be used to treat gingivitis, reduce viral or bacterial load of saliva, treat colds sores, reduction of organisms causing bad breath, act as a preventative or treatment for caries, etc. FIG. 22 shows one embodiment with the preferred shape of the device being that of an upper or lower retainer or both that fits over the teeth. The device can be equipped with a method to determine when it has been inserted properly and when the mouth is closed so that light does not escape and reach the eyes. Alternately, the light dosage could be low enough to not pose a danger to the eyes or it could be directed to the interior of the mouth so that little light would escape. Alternately, the device could have a cover that is on the exterior of the mouth and is applied over it to prevent the escape of light.

The light could have a multiplicity of lights to help evenly spread light throughout the mouth or it could have one light with reflectors, light guides and/or diffusers to distribute the light. Alternately, the light could be applied to one specific area such as the tops of the molars to act as a preventative treatment for caries. In the preferred embodiment the device is used as a prophylaxis to promote general health and it acts to retard or prevent gingivitis, caries, and cold sores. In the preferred embodiment the light can irradiate approximately the entire gum line. In a variant of the preferred embodiment the light can be configured to irradiate only certain problem areas of the gum line. In the preferred embodiment the light is highly pulsed light delivered in less than five seconds using one to one thousand pulses. In the preferred embodiment the retainer is made of a UV transparent material such as TEFLON and may be removable for cleaning or replacement. In the preferred embodiment there can be a light directed to toward the tongue and the back of the mouth to inactivate organisms that cause halitosis. In the preferred embodiment of the light the mouthpiece is fitted into the mouth and has a cable that leads to controls for the device. The controls can verify that the device has been inserted properly by use of light sensors embedded in the mouthpiece, pressure sensors in the mouth piece, proximity detectors, etc. The controls can also verify that the mouthpiece is in proper operating condition. In the preferred embodiment the controls can be set for various conditions including use as a daily prophylaxis with general low level treatment, treatment of cold sores with light directed to the mouth tissues, or treatment of gingivitis with light direct to one or more of the four gum lines.

Most organisms that are in the oral cavity can be effectively inactivated with a dosage of approximately 1 to 20 mj/cm² of UVC. Wide spectrum pulsed light is several times more effective than UVC alone and if it is used to treat an area it is possible that a dose of wide spectrum light with approximately 5 mj/cm² of UVC would be sufficient. At a dosage of less than 20 mj/cm² erythema will be minimal and should not cause a problem. However, if erythema is a concern then a UVB filter can be applied over the lens to reduce erythema. The preferred embodiment of a light used as a prophylaxis treatment for the oral cavity would use a pulsed wide spectrum light delivering a dosage of approximately 5 to 30 mj/cm² per treatment. Preferably the dosage would be delivered in 1 to 100 pulse delivered in one second or less. An alternative embodiment would be a monochromatic LED that delivered a dosage of approximately 5 to 30 mj/cm² of UVC.

Substantially higher dosages could be applied to the teeth to prevent or treat dental caries if the light did not irradiate surrounding tissue. In this case UVC dosages of up to 300 J/cm² could be applied. Wide spectrum pulsed light would be particularly effective in this application since organisms in the oral cavity are adversely impacted by heat which pulsed light can generate to work synergistically with other wavelengths.

Once an organism has caused an oral infection it is necessary to apply sufficient light to penetrate and inactivate the organisms. For very superficial infections the dosage may need to be increases by an order of magnitude to take into account that only about 10% of the light may penetrate thus a dosage of 50 to 300 mj/cm² of UVC may be required. For deeper infections the rate of penetration may be substantially less and in the range of 1% to less than 0.01% which may require a dosage of 500 mj/cm² to 3 J/cm² for 1% percent penetration to 50 to 300 J/cm² for a penetration rate of 0.01%. This dosage could be reduced significantly if pulsed wide spectrum light was used. For example the dosage for 0.01% penetration may be reduced to approximately 10 to 100 J/cm² of UVC or lower if pulsed wide spectrum light is used. If erythema is a concern then a UVB filter can be used to reduce erythema. The preferred embodiment of to treat infections would use a pulsed wide spectrum light delivering a dosage of approximately 500 mj/cm² to 10 J/cm² per treatment or more for severe oral infections. Care must be taken not to damage the sensitive tissues of the mouth, therefore, dosages should be precisely applied only to the area being treated and lower dosages should be tried before high dosages are used.

In a preferred embodiment the mouthpiece is fitted into the mouth and a fiber optic light guide is used to direct light generated outside the mouth to be dispersed inside the mouth. In this preferred embodiment the mouthpiece may still have a detector that shows if the mouthpiece is in proper position and so communicates this to exterior controls. In this preferred embodiment the mouthpiece may be interchangeable so that multiple persons could share one device but have their own mouthpiece. Additionally, specialty mouthpieces could be used for various treatments, such as the malleable (e.g., when heated with hot water), customizable mouthpieces worn by football players. For example, a mouthpiece for treatment of gingivitis might have the light directed to the gum lines while one for cold sores could have light directed to the mouth tissues. In a preferred embodiment the light guide could have a tip that permits spot treatment of areas of the mouth.

In a preferred embodiment there may be a multiplicity of lights aimed in various directions that may be independently controlled and which may be programmed to sequence and/or deliver varying amounts of light to different parts of the mouth.

In a preferred embodiment there may be a multiplicity of lights such as LEDs embedded in the mouthpiece to provide light in desired areas. In a variant of this embodiment the LEDs are a mixture providing both UVC and thermal energy.

In a preferred embodiment the mouthpiece can be put in a container of liquid and then turned on to disinfect the mouthpiece and liquid in the container. In a variant of this embodiment the liquid is water and the light from the mouthpiece generates a small amount of ozone to aid in disinfection. In another variant the container has its own light or lights to aid in disinfection of the mouthpiece and may also generate ozone or other species that can aid in disinfecting the mouthpiece.

In a preferred embodiment the device can monitor the condition of the mouthpiece and recommend replacement when it has deteriorated or has been used too long.

In a preferred embodiment device is self-contained and fits completely in the mouth and uses a supercapacitor to power the light. In a preferred embodiment the unit is recharged wirelessly.

In a preferred embodiment this device could be used to treat mouth cancer and tumors with the area surrounding the cancer masked off and high dosages of light used to inactivate and kill the cancerous cells or tumor cells.

In a preferred embodiment the area surrounding the area to be treated may be masked and the mask may remain in place for several treatments. The mask may have a medication that decreases any pain associated with the treatment either during or after the treatment and it may be left in place for a period of time as necessary.

A variant of the preferred embodiment may have the light generated exterior to the mouth and transmitted inside by a light guide. Alternately, the power to generate the light could be external while the light itself was inside the oral cavity. Other variants of the preferred embodiment can use point treatment instead of a widely distributed light, different types and sizes of lights, continuous and pulsed lights and combinations thereof, and adjunct treatments such as heat and ultrasound.

The embodiments of this device may be incorporated into other device embodiments disclosed in this application or other devices that one skilled in the art could develop.

### Preferred Embodiment for Internal Treatment (Tumors, Cancer, Treatment During Surgery, Wounds)

The preferred embodiments are descriptions of possible combinations of features and are illustrative, not exhaustive. One skilled in the art could develop similar combinations that include or exclude certain items and these combinations are contemplated as part of this invention.

The preferred embodiment of the device used to treat interior portions of the body would comprise one or more light guides that could be used to see the area being treated while also being capable of transmitting the light used for treatment. In the preferred embodiment, very high light doses could be applied in areas that were not as sensitive. In the preferred embodiment the device could be left in place for a period of time to provide multiple treatments. In another variant the delivery light guide could also have a small channel for delivery of a medication or solution. The solution could be photodynamically activated or it could be active in itself The light guide could also have sensor embedded to measure temperature, light dose, and other important parameters.

A preferred embodiment of the device could have multiple fiber optic tips at the end of the guide that can penetrate in a number of areas thus reducing the distance that the treatment light must penetrate into the area being treated. This embodiment could be of particular use for treatment of tumors and cancer.

Another preferred embodiment would be a light to treat exposed areas of the body during surgery. The light could be applied before, during, or after surgery or a combination thereof. This type of light could also be used to prepare the body and an organ that was being removed from a donor for transplantation or was being inserted into a recipient. In this embodiment a very quick, intense, pulsed, wide spectrum light could inactivate undesirable organisms and also reduce the chance of an organ being rejected.

Most organisms can be effectively inactivated with a dosage of approximately 1 to 20 mj/cm² of UVC. Wide spectrum pulsed light is several times more effective than UVC alone and if it is used to treat an area it is possible that a dose of wide spectrum light with approximately 5 mj/cm² of UVC would be sufficient. The preferred embodiment of a light used to prevent infections on the surface of tissues would use a pulsed wide spectrum light delivering a dosage of approximately 5 to 30 mj/cm² per treatment. Preferably the dosage would be delivered in 1 to 100 pulse delivered in one second or less. An alternative embodiment would be a monochromatic LED that delivered a dosage of approximately 5 to 30 mj/cm² of UVC.

Once an organism has caused an infection it is necessary to apply sufficient light to penetrate and inactivate the organisms. For very superficial infections the dosage may need to be increases by an order of magnitude to take into account that only about 10% of the light may penetrate thus a dosage of 50 to 300 mj/cm² of UVC may be required. For deeper infections the rate of penetration may be substantially less and in the range of 1% to less than 0.01% which may require a dosage of 500 mj/cm² to 3 J/cm² for 1% percent penetration to 50 to 300 J/cm² for a penetration rate of 0.01%. This dosage could be reduced significantly if pulsed wide spectrum light was used. For example the dosage for 0.01% penetration may be reduced to approximately 10 to 100 J/cm² of UVC or lower if pulsed wide spectrum light is used. The preferred embodiment of to treat infections would use a pulsed wide spectrum light delivering a dosage of approximately 500 mj/cm² to 10 J/cm² per treatment or more for severe internal infections.

In the preferred embodiment dosage ranges to treat internal infections, diseases, and disorders would be approximately the same as for treatment of other skin diseases and disorders and could range from 10 mj/cm² to up to 300 J/cm² of UVC. Use of pulsed wide spectrum light can significantly decrease the dosage required.

Treatment of tumors and certain other internal disorders may require significantly higher dosages of 300 J/cm² of UVC per treatment or higher. As a whole though the dosages used to treat skin infections are a good beginning guide to practitioners using germicidal light to treat internal infections, diseases, and disorders.

In another preferred embodiment the device could be used to disinfect and sterilize tissue, bone, and artificial implants. In particular, the device could be used for tissue and bones that may carry MRSA organisms.

Another preferred embodiment of the device uses an optically transparent dressing over a wound and germicidal light to both kill organisms on the surface and inside tissue and to also stimulate blood circulation and other bodily functions that aid in healing. In a preferred embodiment the wound is cleaned prior to application of the dressing and the dressing is kept in place for several treatments.

In another preferred embodiment the device may be made small enough to fit through a catheter to provide treatment to a specific area. In a variant of this embodiment the device may operate as it is being drawn through the catheter to keep the catheter free of colonization by infectious organisms.

The embodiments of this device may be incorporated into other device embodiments disclosed in this application or other devices that one skilled in the art could develop.

### Preferred Embodiment of Goggles Worn During Treatment

The preferred embodiments are descriptions of possible combinations of features and are illustrative, not exhaustive. One skilled in the art could develop similar combinations that include or exclude certain items and these combinations are contemplated as part of this invention.

Many events or actions or activities require special protection of the eyes. This invention is a method and device to protect eyes during said events, actions, and activities. The invention consists of an eye covering (“goggles” hereinafter, but may refer to any eye covering) which can be used to protect the eyes. Any shield such as glasses or a mask is also included specifically as part of this invention.

The goggles have 1) a way to positively indicate that they are properly fitted on a person and/or 2) the goggles have a positive way to prevent operation of a device (or start an activity, action, or event) without the goggles being properly located or fitted.

FIG. 22 shows one preferred embodiment of the goggles that may use is a detector to determine proper fitting that indicates when they are on a person (for example a light detector that notes that the eye is blocking light from the open end of the goggles, or a proximity detector that shows something is in the goggles, or an infrared detector showing a heat source (the eyes) are in the goggles, or a combination of all or some different means). Another method that could be used to identify that the goggles are on correctly are pressure switches embedded in foam on the perimeter of the goggles. Capacitance switches and other similar devices could also be used to ensure the goggles are on correctly.

In another embodiment the goggles will have a small retinal scanner. The scanner can both verify the goggles have been correctly placed and can also identify the person wearing them. This identification could be used to provide a certain treatment regimen or it could be used to prevent a person from receiving more treatments than are desirable. This scanner can be interfaced and interlocked with treatment units and it can also provide information to a system that keeps historical track of the treatments provided. The retinal scanner can be linked with software to identify the person by name which can serve as a cross check to the person's ID bracelet or other form of identification.

FIG. 23 shows a preferred embodiment where the goggles, when properly in place, can also emit a signal that permits the phototherapy device to be operated (and can also signal a certain dose per treatment time or per day). The goggles can be constructed to let through some light so a person could see somewhat but block treatment light, such as UV light (and maybe some other light if it is a light detector that shows they are on properly). For example, if the goggles filtered out blue light in addition to UV as long as the photodetector inside the goggles detected blue light it would be an indication that the goggles were not on correctly

In a preferred embodiment that goggles (eye coverings) could have an external indicator to show they are on properly. For example there could be a small green LED that lights up when each eyepiece is on correctly. The indicator could send a wireless signal that could be interlocked with equipment. The wireless signal could give additional information about whether the goggles are on correctly. For example, each eyepiece could have two light detectors and two infrared detectors. For the eyepiece to be in correct position each of these would need to show they were operating correctly and read as they should. However, each of these detectors could broadcast individual information and this could be monitored and thresholds adjusted as desired. For example, the light detectors could be set to be ‘green’ if the light level was 0.1 lumens or less for normal ‘go’ operation. However, the operator could see the actual level and perhaps if it was 0.2 lumens he might override the detector or reset the threshold to 0.25 lumens. Also, if one of the two light detectors was out you could use software to disable its interlock.

This invention would work for glasses as well as goggles or for any other eye covering.

This invention could be used for swimming goggles where it would be especially useful for making sure small children had goggles on right.

The external signal could be a 4-20 ma or some other signal that was delivered could be transferred via a wire or fiber.

In an embodiment that goggles could be quickly put on, which would be good for emergency use. They could provide feedback (for example, emitting a sound until they are properly adjusted—perhaps a buzz that gets louder as it is positioned right and a beep when it is fully in position) so a person could put them on and adjust them correctly even with his eyes closed.

In a preferred embodiment the goggles have a means to detect if they are properly installed. The means may be an infrared detector that ensures that there is a thermal mass such as an eye close to the goggles. The means may be a proximity indicator showing a mass is next to the goggles. The means may also be a light detector that indicates when the light has substantially decreased (due to the goggles being put on and light being block when the goggles seal against the skin). The means may be a some kind of capacitance indicator that notes that substantially all of the perimeter of the goggles is in contact with the skin. The means may be a combination of the above or may be one of a number of means to ensure that the goggles are on correctly. All these means are contemplated for this invention.

In a preferred embodiment the goggles have a means to indicate that they are installed properly. The means may be a visual indicator such as a green light indicating that the goggles are installed correctly. The indication could be inside the goggles (to alert the person they are on that they are installed properly) or it could be external or it could be both.

In a preferred embodiment the goggles may have the means to emit signal indicating they are on correctly. This signal could be sent out via a cable or wirelessly. The signal may have the ability to interface with a treatment device to prevent the device from working until the signal indicates proper installation. In a variant of the preferred embodiment more than one set of goggles may be interlocked with a device to prevent treatment if they are not properly installed. For example, the person treated and the person administering the treatment may need to have goggles on correctly to permit a device to operate.

In a preferred embodiment a retinal scanner is embedded in the goggles to identify that they are installed correctly and to identify the person who is receiving treatment.

In a preferred embodiment an RFID chip or other device is embedded or attached to the goggles. The RFID chip can provide positional information to ensure that the goggles have been put on correctly.

In another preferred embodiment the device is similar to the first embodiment but has screens inside that can be linked to cameras so the external area can be seen by the wearer of the goggles

In another preferred embodiment the device is similar to the first embodiment but has a screen indicating pertinent information such as how long is left in the treatment regimen

In a another preferred embodiment the goggles may not screen out all light. They may darken the area or only screen out harmful light thus permitting the wearer to see through the goggles.

In another preferred embodiment the goggles may be interlocked with a device that plugs into a power source and then in turn can be plugged into by another device. The interlock would then permit power to proceed to the device that was plugged in if the goggles were in the proper position.

In another further embodiment the device is similar to a pair of glasses and does not block out all of the light but screens out enough to provide for safe treatment, or may block light only during the treatment period by mechanical or electrical means.

In another further embodiment the device is similar to a face shield used in welding and protects the entire face and not just the eyes.

In another further embodiment the device could be made to be disposable, for example heavy paper goggles with translucent plastic for lenses. A variant of this embodiment would be for part of the goggles to be disposable, for example the part that contacts the skin could be made of a foam that can be applied easily to the goggles and then removed and discarded after use. The disposable portion of the goggle could have an RFID chip to identify the use of the goggle (i.e. a onetime use chip that permits the device to be operated).

In another further embodiment the device is similar to a binocular eyepiece and the eyes would be positioned to cover said eyepieces. The eyepieces could be attached to a larger device or integrated into a mask. This embodiment would permit precise positioning of the face along with the eyes and could be used to enhance treatment. This embodiment could also be included with the a retinal scanner to further identify the person being treated.

The embodiments of this device may be incorporated into other device embodiments disclosed in this application or other devices that one skilled in the art could develop.

### 7 SUMMARY, RAMIFICATIONS, AND SCOPE

Accordingly, this invention can be used to prevent and treat a wide variety of ailments. It has the following advantages over the current method of treatments:
With respect to treatment using oral medications, the invention eliminates unwanted and potentially dangerous side effects that such medications can cause.
With respect to treatment using oral medications, the invention uses a very small number of treatments (one to perhaps a dozen) to eliminate the infection while medications must be taken continuously for a time.
With respect to treatment using topical medications, the proposed treatment has the potential to have fewer side effects.
With respect to other treatments used for existing infections, this treatment can also be used periodically to prevent infections from becoming established.

Although the descriptions above contain many specificities, these should not be construed as limiting the scope of the invention but merely as providing illustrations of some of the presently preferred embodiments of this invention. Any of the features of one embodiment may be used with any of the features of other embodiments or without all of the features of some or all embodiments.

Thus the scope of this invention should be determined by the appended claims and their legal equivalents, rather than by the examples given.

We claim:

1. A light device for the prevention or treatment of an infection, disease, or disorder comprising:
a housing having a source of germicidal light;
and means for applying at least one fixed dosage of germicidal light to a patient.
an enclosure within said housing communicating with said germicidal light for receiving an area to be irradiated; and
a controller for selectively controlling irradiation of said enclosure and area within said housing by said germicidal light for a predetermined dosage.

2. The light device of claim 1, further comprising:
a control system for said light device for delivering at least one predetermined dosage of germicidal light in the range of 10 mJ/cm² to 100 J/cm² of UVC per treatment.

3. The light device of claim 1, further comprising:
a light sensor for sensing light entering from the enclosure from outside of the light device,
a shut off device for preventing operation of the light device when the light sensors senses light entering from the outside of the light device.

4. The light device of claim 1, further comprising:
a proximity detector that electrically in communication with the light device and prevents the device from operating when guard remote from the light device is not sensed to be within a predetermined range of the light device.

5. The light device of claim 1, further comprising:
a safety device remote from the light device for detecting light escaping from the enclosure to the safety device;
a shut off device for preventing operation of the light device when the safety device senses light escaping to the outside of the light device.

6. A light device comprising:
A source of germicidal light;
A hand unit that can be held in the hand to deliver said germicidal light to an applied light to a desired area;
A tip on said hand unit for aiming the germicidal light for shielding the light;
a lens on said tip for focusing said germicidal light on said desired area;
a filter between said germicidal light and said desired area for selectively removing unwanted wavelengths of light from the applied light;

7. A light device for the prevention or treatment of an infection, disease, or disorder of a user comprising:
a housing having a source of germicidal light;
and an mask sensor for detecting the positioning of the mask on the user being treated;
a shut off device to prevent the device from operating if a the mask senses that the device is not properly positioned.

8. A light device for the prevention or treatment of an infection, disease, or disorder of the mouth comprising:
a housing having a source of germicidal light;
a mouthpiece that for receiving the teeth of a user within the mouthpiece;
a light broadcasting device on said mouthpiece for irradiating an area within the mouth.

9. A light device for the prevention or treatment of an infection, disease, or disorder of a nasal passageway comprising:
a housing having a source of germicidal light for pulsed at least one time;
and a tip sized to fit inside a nostril to irradiate the nares.

10. A light device for the prevention an infection, disease, or disorder from a puncture wound comprising:
a glucose monitor for sampling having a probe for puncturing a user;
a germicidal light mounted on the glucose monitor for irradiating an area to be punctured to prevent infection, disease or disorder at the puncture are.

11. A light device for the prevention or treatment of an infection, disease, or disorder comprising a germicidal light with a pulse width of 1 femtosecond to 50 milliseconds that the pulses produce light in the ultraviolet and visible range between 200 nm and 700 nm of which at least 1% is in the range of 200 nm to 280 nm and 10% is in the range of 200 nm to 400 nm and delivering at dosage of at least 10 mj/cm² of light in the range of 200 nm to 280 nm.
